(19) United States
(12) Patent Application Publication
(10) Pub. No.: US 2023/0104358 A1
(43) Pub. Date: Apr. 6, 2023

(54) Refillable Implantable Device for Delivering a Drug Compound

(71) Applicant: Celanese EVA Performance Polymers LLC, Irving, TX (US)

(72) Inventors: Vijay Gyanani, Powell, OH (US); Jeffrey Haley, Norwood, OH (US); Keith Henry, Middleton, DE (US); Kevin Welsh, Worthville, KY (US)

(21) Appl. No.: 17/948,277

(22) Filed: Sep. 20, 2022

Related U.S. Application Data
(60) Provisional application No. 63/305,772, filed on Feb. 2, 2022.
(60) Provisional application No. 63/246,844, filed on Sep. 22, 2021.

(51) Int. Cl. A61M 31/00; A61K 9/00; A61M 37/00; A61K 31/573; A61K 31/663

(52) U.S. Cl. CPC A61M 31/002; A61K 9/0024; A61K 9/0039; A61K 31/573; A61K 31/663; A61M 37/0069

(57) ABSTRACT

An implantable and refillable device for delivering a drug compound is provided. The device comprises a reservoir within which the drug compound is capable of being retained, wherein the reservoir defines a first surface and a second surface opposing the first surface. A release structure comprising a hydrophobic polymer surrounds at least a portion of the reservoir. The release structure is in communication with the reservoir such that the drug compound can pass from the reservoir through the release structure. A septum is positioned adjacent to the first surface of the reservoir and a backing layer is positioned adjacent to the second surface of the reservoir.

### RELATED APPLICATIONS

The present application is based upon and claims priority to U.S. Provisional Pat. Application Serial No. 63/246,844, having a filing date of Sep. 22, 2021 and U.S. Provisional Pat. Application Serial No. 63/305,772, having a filing date of Feb. 2, 2022, which are incorporated herein by reference.

### BACKGROUND

Implantable medical devices have been used for some time to provide long-term release of drug compounds. Unfortunately, many implantable devices can carry risks and complications including dosing errors, particularly for drug compounds that have been traditionally difficult to controllably deliver over a sustained period of time. Attempts have been made to make implantable delivery devices refillable for extended controlled delivery of drug compounds. This has been met with difficulties as well, including infection arising from the port system used for refilling the device reservoir and an inconsistent drug delivery rate due to the need for multiple device refilling cycles. As such, a need continues to exist for an implantable and refillable delivery device that is capable of delivering a drug compound over a sustained period of time.

### SUMMARY OF THE INVENTION

In accordance with one embodiment of the present disclosure, an implantable and refillable device is disclosed. The device a reservoir within which a drug compound is capable of being retained, wherein the reservoir defines a first surface and a second surface opposing the first surface. A release structure comprising a hydrophobic polymer surrounds at least a portion of the reservoir. The release structure is in communication with the reservoir such that the drug compound can pass from the reservoir through the release structure. Further, a septum is positioned adjacent to the first surface of the reservoir and a backing layer is positioned adjacent to the second surface of the reservoir.

Other features and aspects of the present disclosure are set forth in greater detail below.

### BRIEF DESCRIPTION OF THE DRAWINGS

A full and enabling disclosure of the present disclosure, including the best mode thereof, directed to one of ordinary skill in the art, is set forth more particularly in the remainder of the specification, which makes reference to the appended drawings in which:

FIG. 1 is a perspective view of one embodiment of an implantable medical device of the present disclosure;

FIG. 2 is a cross-sectional view of the implantable medical device of FIG. 1;

FIG. 3 is a perspective view of another embodiment of an implantable medical device of the present disclosure;

FIG. 4 is a cross-sectional view of the implantable medical device of FIG. 3; and

FIG. 5 is a cross-sectional view of a multi-layered septum as may be included in a device as disclosed herein.

Repeat use of references characters in the present specification and drawing is intended to represent same or analogous features or elements of the disclosure.

### DETAILED DESCRIPTION

It is to be understood by one of ordinary skill in the art that the present discussion is a description of exemplary embodiments only, and is not intended as limiting the broader aspects of the present disclosure.

Generally speaking, the present disclosure is directed to an implantable device that is capable of delivering a drug compound to a patient (e.g., human, pet, farm animal, etc.) over a sustained period of time to help prohibit and/or treat a condition, disease, and/or cosmetic state of the patient. The device includes a reservoir within which the drug compound is capable of being retained and a release structure that is in communication with the reservoir such that the drug compound can pass from the reservoir through the release structure. The device also includes a septum that can be used for refilling the reservoir of the device and a backing layer opposite the septum that can inhibit needle penetration through the device during refilling.

The implantable device may have a variety of different geometric shapes, such as circular or ovoid disc, cylindrical (rod), ring, doughnut, helical, elliptical, triangular, ovular, etc. In one embodiment, for example, the device may have a generally circular cross-sectional shape so that the overall structure is in the form of a disc or rod. In such embodiments, the device will typically have a diameter of from about 0.5 to about 3 centimeters, such as from about 0.75 to about 2 centimeters, in some embodiments from about 0.8 to about 1.5 centimeters. The height of the device may vary, but is typically in the range of from about 0.25 to about 1.5 centimeters. In some embodiments, a reservoir of a device can define a volume of from about 0.05 to about 5 milliliters, for instance from about 1 to about 4 milliliters. Regardless of the particular size or shape, the device includes a release structure adjacent to the reservoir and through which one or more drug compounds can pass.

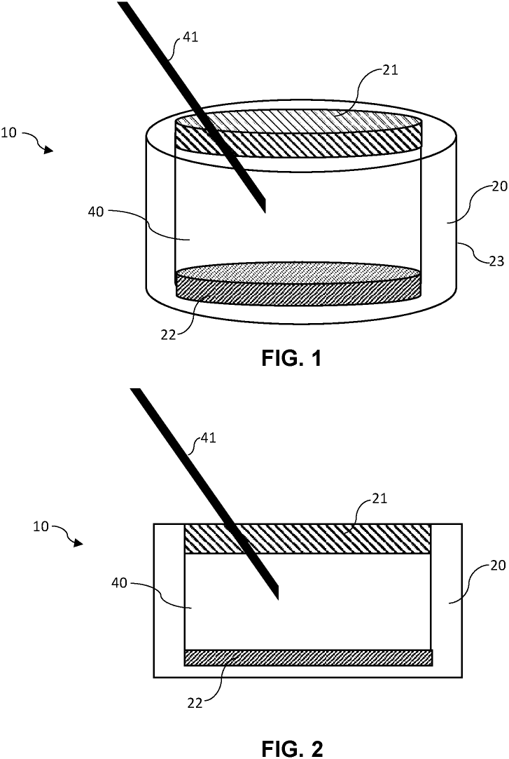

Referring to FIG. 1 and FIG. 2, for example, one embodiment of an implantable device 10 is shown that contains a reservoir 40 having a generally circular cross-sectional shape and that is generally disc-shaped in nature. A release structure 20 likewise surrounds at least a portion of the reservoir 40, and in some cases, the entire reservoir 40. The device also includes a septum 21 that is positioned adjacent to a first surface (e.g., upper surface) of the reservoir 40 and a backing layer 22 that is positioned adjacent to a second opposing surface (e.g., lower surface) of the reservoir 40. As shown, the release structure 20 may also surround at least a portion of the septum 21 and/or at least a portion of the backing layer 22. During use of the device 10, the drug compound is capable of being released from the reservoir 40 by passage through the release structure 20 so that it exits from an external surface 23 of the device. When it is desired to refill the device, a needle 41 can penetrate the septum and be inhibited by the backing layer 22 from over-penetration and passage completely through and out of the reservoir. The drug compound can thus be injected into the reservoir for refilling.

Of course, in other embodiments, the release structure may contain multiple layers. In the device of FIG. 1 and FIG. 2, for example, one or more additional layers (not shown) may be disposed over the layer 20 to help further control release of the drug compound.

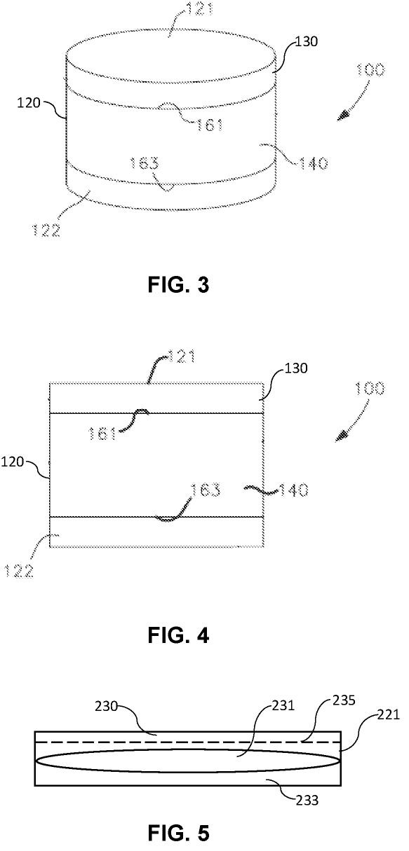

The device may be configured so that the backing layer is held within the release structure or alternatively forms an outer surface of the device. Referring to FIG. 3 and FIG. 4, for example, one embodiment of an implantable device 100 is shown that contains a reservoir 140 having a generally circular cross-sectional shape and a height so that the resulting device is generally disc-shaped in nature. The reservoir 140 is surrounded by a release structure 120 that has an upper edge 161 to which is adhered a septum 121 and a lower edge 163 to which is adhered a backing layer 122. As shown, the septum 121 and the backing layer 122 can have a generally circular cross-sectional shape and form an upper and lower surface of the device 100. If desired, a portion of the release structure 120 may also extend beneath a lower surface of the septum 121 and along an upper surface of the backing layer 122, which can improve the seal between the materials and prevent any uncontrolled leakage of the contents of the reservoir 140. During use of the device 100, the drug compound is capable of being released from the reservoir 140 and through the release structure 120 so that it exits from the device. Of course, if desired, one or more additional layers (not shown) may also be disposed over the release structure 120 to help further control release of the drug compound.

Through selective control over the particular nature of the release structure, septum, and/or backing layer, as well as other aspects of the device, it is believed that the resulting device can be effective for sustained release of one or more drug compounds over a prolonged period of time. For example, upon a single filling of the reservoir, the implantable device can release the drug compound for a time period of about 5 days or more, in some embodiments about 10 days or more, in some embodiments from about 20 days to about 60 days, and in some embodiments, from about 25 days to about 50 days (e.g., about 30 days) or even longer in some embodiments. For instance, a device need only be refilled on a month-long time scale, e.g., once a month, bimonthly, once in three months, once in four months, once in six months, or more. In some embodiments, a device can be designed to be refilled once a year, or even longer.

Through refilling of the reservoir, the implantable device can release the drug compound from several weeks to several months in some embodiments. For instance, the effective drug delivery time period over the entire course of use (i.e., including refilling of the device) can be in the range of about three months to multiple years, e.g., about six months to about six years; about six months to about five years; or about one year to about four years, such as about 18 months in some embodiments. Further, the drug compound can also be released in a controlled manner (e.g., zero order or near zero order) over the course of the release time period. Moreover, through selective control over the particular nature of the release structure, septum, and/or backing materials used in forming the device, the device can be refilled multiple times without loss of desirable release characteristics.

Various embodiments of the present disclosure will now be described in more detail.

### I. Release Structure

As indicated above, the device includes a release structure through which the drug compound can be delivered from a reservoir to a surrounding area, e.g., via passive diffusion through the release structure. The release structure generally contains a polymeric matrix that is formed from at least one polymer that is generally hydrophobic in nature so that it can retain its structural integrity for a certain period of time when placed in an aqueous environment, such as the body of a mammal, and stable enough to be stored for an extended period before use. Examples of suitable hydrophobic polymers for this purpose may include, for instance, silicone polymer, polyolefins, polyvinyl chloride, polycarbonates, polysulphones, styrene acrylonitrile copolymers, polyurethanes, silicone polyether-urethanes, polycarbonate-urethanes, silicone polycarbonate-urethanes, etc., as well as combinations thereof. Of course, hydrophilic polymers that are coated or otherwise encapsulated with a hydrophobic polymer are also suitable for use in the release structure polymer matrix. Typically, the melt flow index of the hydrophobic polymer ranges from about 0.2 to about 100 g/10 min, in some embodiments from about 5 to about 90 g/10 min, in some embodiments from about 10 to about 80 g/10 min, and in some embodiments, from about 30 to about 70 g/10min, as determined in accordance with ASTM D1238-20 at a temperature of 190° C. and a load of 2.16 kilograms.

In certain embodiments, the release structure may contain a semicrystalline olefin copolymer. The melting temperature of such an olefin copolymer may, for instance, range from about 40° C. to about 140° C., in some embodiments from about 50° C. to about 125° C., and in some embodiments, from about 60° C. to about 120° C., as determined in accordance with ASTM D3418-15. Such copolymers are generally derived from at least one olefin monomer (e.g., ethylene, propylene, etc.) and at least one polar monomer that is grafted onto the polymer backbone and/or incorporated as a constituent of the polymer (e.g., block or random copolymers). Suitable polar monomers include, for instance, a vinyl acetate, vinyl alcohol, maleic anhydride, maleic acid, (meth)acrylic acid (e.g., acrylic acid, methacrylic acid, etc.), (meth)acrylate (e.g., acrylate, methacrylate, ethyl acrylate, methyl methacrylate, ethyl methacrylate, etc.), and so forth. A wide variety of such copolymers may generally be employed in the polymer composition, such as ethylene vinyl acetate copolymers, ethylene (meth)acrylic acid polymers (e.g., ethylene acrylic acid copolymers and partially neutralized ionomers of these copolymers, ethylene methacrylic acid copolymers and partially neutralized ionomers of these copolymers, etc.), ethylene (meth)acrylate polymers (e.g., ethylene methylacrylate copolymers, ethylene ethyl acrylate copolymers, ethylene butyl acrylate copolymers, etc.), and so forth. Regardless of the particular monomers selected, certain aspects of the copolymer can be selectively controlled to help achieve the desired release properties. For instance, the polar monomeric content of the copolymer may be selectively controlled to be within a range of from about 10 wt.% to about 60 wt.%, in some embodiments about 20 wt.% to about 60 wt.%, and in some embodiments, from about 25 wt.% to about 50 wt.%. Conversely, the olefin monomeric content of the copolymer may likewise be within a range of from about 40 wt.% to about 90 wt.%, in some embodiments about 40 wt.% to about 80 wt.%, and in some embodiments, from about 50 wt.% to about 75 wt.%.

In one particular embodiment, for example, the release structure may contain at least one ethylene vinyl acetate polymer, which is a copolymer that is derived from at least one ethylene monomer and at least one vinyl acetate monomer. In certain cases, the present inventors have discovered that certain aspects of the copolymer can be selectively controlled to help achieve the desired release properties. For instance, the vinyl acetate content of the copolymer may be selectively controlled to be within a range of from about 10 wt.% to about 60 wt.%, in some embodiments from about 20 wt.% to about 60 wt.%, in some embodiments from about 25 wt.% to about 50 wt.%, in some embodiments from about 30 wt.% to about 48 wt.%, and in some embodiments, from about 35 wt.% to about 45 wt.% of the copolymer. Conversely, the ethylene content of the copolymer may likewise be within a range of from about 40 wt.% to about 90 wt.%, in some embodiments from about 40 wt.% to about 80 wt.%, in some embodiments from about 50 wt.% to about 75 wt.%, in some embodiments from about 50 wt.% to about 80 wt.%, in some embodiments from about 52 wt.% to about 70 wt.%, and in some embodiments, from about 55 wt.% to about 65 wt.%. The melt flow index of the ethylene vinyl acetate copolymer(s) and resulting polymer matrix may also range from about 0.2 to about 400 g/10 min, in some embodiments from about 1 to about 200 g/10 min, in some embodiments from about 5 to about 90 g/10 min, in some embodiments from about 10 to about 80 g/10 min, and in some embodiments, from about 30 to about 70 g/10 min, as determined in accordance with ASTM D1238-20 at a temperature of 190° C. and a load of 2.16 kilograms. The density of the ethylene vinyl acetate copolymer(s) may also range from about 0.900 to about 1.00 gram per cubic centimeter (g/cm³), in some embodiments from about 0.910 to about 0.980 g/cm³, and in some embodiments, from about 0.940 to about 0.970 g/cm³, as determined in accordance with ASTM D1505-18. Particularly suitable examples of ethylene vinyl acetate copolymers that may be employed include those available from Celanese under the designation ATEVA® (e.g., ATEVA® 4030AC); Dow under the designation ELVAX® (e.g., ELVAX® 40W); and Arkema under the designation EVATANE® (e.g., EVATANE 40-55). In embodiments, the ethylene vinyl acetate copolymer in the release structure polymer matrix is from about 20 wt.% to about 90 wt.%, such as from about 30 wt.% to about 80 wt.%, such as from about 40 wt.% to about 70 wt.%.

Any of a variety of techniques may generally be used to form the ethylene vinyl acetate copolymer(s) with the desired properties as is known in the art. In one embodiment, the polymer is produced by copolymerizing an ethylene monomer and a vinyl acetate monomer in a high pressure reaction. Vinyl acetate may be produced from the oxidation of butane to yield acetic anhydride and acetaldehyde, which can react together to form ethylidene diacetate. Ethylidene diacetate can then be thermally decomposed in the presence of an acid catalyst to form the vinyl acetate monomer. Examples of suitable acid catalysts include aromatic sulfonic acids (e.g., benzene sulfonic acid, toluene sulfonic acid, ethylbenzene sulfonic acid, xylene sulfonic acid, and naphthalene sulfonic acid), sulfuric acid, and alkanesulfonic acids, such as described in U.S. Pat. Nos. 2,425,389 to Oxlev et al.; 2,859,241 to Schnizer; and 4,843,170 to Isshiki et al. The vinyl acetate monomer can also be produced by reacting acetic anhydride with hydrogen in the presence of a catalyst instead of acetaldehyde. This process converts vinyl acetate directly from acetic anhydride and hydrogen without the need to produce ethylidene diacetate. In yet another embodiment, the vinyl acetate monomer can be produced from the reaction of acetaldehyde and a ketene in the presence of a suitable solid catalyst, such as a perfluorosulfonic acid resin or zeolite.

In certain embodiments, it may also be desirable to employ blends of an ethylene vinyl acetate copolymer and another hydrophobic polymer such that the overall blend and release structure have a melting temperature and/or melt flow index within the range noted above. For example, the release structure may contain a first ethylene vinyl acetate copolymer and a second ethylene vinyl acetate copolymer having a melting temperature that is greater than the melting temperature of the first copolymer. The second copolymer may likewise have a melt flow index that is the same, lower, or higher than the corresponding melt flow index of the first copolymer. The first copolymer may, for instance, have a melting temperature of from about 20° C. to about 60° C., in some embodiments from about 25° C. to about 55° C., and in some embodiments, from about 30° C. to about 50° C., such as determined in accordance with ASTM D3418-15, and/or a melt flow index of from about 40 to about 900 g/10 min, in some embodiments from about 50 to about 500 g/10 min, and in some embodiments, from about 55 to about 250 g/10 min, as determined in accordance with ASTM D1238-20 at a temperature of 190° C. and a load of 2.16 kilograms. The second copolymer may likewise have a melting temperature of from about 50° C. to about 100° C., in some embodiments from about 55° C. to about 90° C., and in some embodiments, from about 60° C. to about 80° C., such as determined in accordance with ASTM D3418-15, and/or a melt flow index of from about 0.2 to about 55 g/10 min, in some embodiments from about 0.5 to about 50 g/10 min, and in some embodiments, from about 1 to about 40 g/10 min, as determined in accordance with ASTM D1238-20 at a temperature of 190° C. and a load of 2.16 kilograms. The first copolymer may constitute from about 20 wt.% to about 80 wt.%, in some embodiments from about 30 wt.% to about 70 wt.%, and in some embodiments, from about 40 wt.% to about 60 wt.% of the polymer matrix, and the second copolymer may likewise constitute from about 20 wt.% to about 80 wt.%, in some embodiments from about 30 wt.% to about 70 wt.%, and in some embodiments, from about 40 wt.% to about 60 wt.% of the release structure.

In certain cases, ethylene vinyl acetate copolymer(s) constitute the entire hydrophobic polymer content of the release structure. In other cases, however, it may be desired to include other polymers, such as other hydrophobic polymers. When employed, it is generally desired that such other hydrophobic polymers constitute from about 0.001 wt.% to about 30 wt.%, in some embodiments from about 0.01 wt.% to about 20 wt.%, and in some embodiments, from about 0.1 wt.% to about 10 wt.% of the hydrophobic polymer content of the release structure polymer matrix. In such cases, ethylene vinyl acetate copolymer(s) may constitute about from about 70 wt.% to about 99.999 wt.%, in some embodiments from about 80 wt.% to about 99.99 wt.%, and in some embodiments, from about 90 wt.% to about 99.9 wt.% of the hydrophobic polymer content of the release structure.

When employing multiple release structure layers, it is typically desired that each layer contains a polymer matrix that includes a hydrophobic polymer. For example, a first release structure layer may contain a first polymer matrix and a second release structure layer may contain a second polymer matrix. In such embodiments, the first and second polymer matrices each contain a hydrophobic polymer, which may be the same or different. In one embodiment, for instance, all of the release structure layer(s) employ the same hydrophobic polymer (e.g., α-olefin copolymer). In yet other embodiments, release structure layers may employ different hydrophobic polymers, for instance a first release structure layer may employ a hydrophobic polymer (e.g., α-olefin copolymer) that has a lower melt flow index than a polymer employed in a second release structure layer. Among other things, this can further help control the release of the drug compound from the device.

The release structure may also optionally contain one or more excipients as described above, such as radiocontrast agents, bulking agents, plasticizers, surfactants, crosslinking agents, flow aids, colorizing agents (e.g., chlorophyll, methylene blue, etc.), antioxidants, stabilizers, lubricants, other types of antimicrobial agents, preservatives, etc. to enhance properties and processability. When employed, the optional excipient(s) typically constitute from about 0.01 wt.% to about 60 wt.%, and in some embodiments, from about 0.05 wt.% to about 50 wt.%, and in some embodiments, from about 0.1 wt.% to about 40 wt.% of a release structure layer.

To help control the release rate from the implantable medical device, a hydrophilic compound may also be incorporated into the release structure that is soluble and/or swellable in water. The weight ratio of the hydrophobic polymers, e.g., ethylene vinyl acetate copolymer(s), to the hydrophilic compounds within the release structure may range about 0.25 to about 200, in some embodiments from about 0.4 to about 80, in some embodiments from about 0.8 to about 20, in some embodiments from about 1 to about 16, and in some embodiments, from about 1.2 to about 10. Such hydrophilic compounds may, for example, constitute from about 1 wt.% to about 60 wt.%, in some embodiments from about 2 wt.% to about 50 wt.%, and in some embodiments, from about 5 wt.% to about 40 wt.% of the polymer matrix, while hydrophobic polymers typically constitute from about 40 wt.% to about 99 wt.%, in some embodiments from about 50 wt.% to about 98 wt.%, and in some embodiments, from about 60 wt.% to about 95 wt.% of a release structure layer.

Suitable hydrophilic compounds may include, for instance, polymers, non-polymeric materials (e.g., glycerin, saccharides, sugar alcohols, salts, etc.), etc. Examples of suitable hydrophilic polymers include, for instance, sodium, potassium and calcium alginates, carboxymethylcellulose, agar, gelatin, polyvinyl alcohols, polyalkylene glycols (e.g., polyethylene glycol), collagen, pectin, chitin, chitosan, poly-1-caprolactone, polyvinylpyrrolidone, poly(vinylpyrrolidone-co-vinyl acetate), polysaccharides, hydrophilic polyurethane, polyhydroxyacrylate, dextran, xanthan, hydroxypropyl cellulose, methylcellulose, proteins, ethylene vinyl alcohol copolymers, water-soluble polysilanes and silicones, water-soluble polyurethanes, etc., as well as combinations thereof. Particularly suitable hydrophilic polymers are polyalkylene glycols, such as those having a molecular weight of from about 100 to 500,000 grams per mole, in some embodiments from about 500 to 200,000 grams per mole, and in some embodiments, from about 1,000 to about 100,000 grams per mole. Specific examples of such polyalkylene glycols include, for instance, polyethylene glycols, polypropylene glycols polytetramethylene glycols, polyepichlorohydrins, etc.

Optionally, the release structure can include a plurality of water-soluble particles distributed within the polymer matrix. The particle size of the water-soluble particles can be controlled to help achieve the desired delivery rate. For instance, the median diameter (D₅₀) of the particles can be about 100 micrometers or less, in some embodiments about 80 micrometers or less, in some embodiments about 60 micrometers or less, and in some embodiments, from about 1 to about 40 micrometers, such as may be determined using a laser scattering particle size distribution analyzer (e.g., LA-960 from Horiba). The particles may also have a narrow size distribution such that 90% or more of the particles by volume (D₉₀) have a diameter within the ranges noted above. In addition to controlling the particle size, the materials employed to form the water-soluble particles can also be selected to achieve the desired release profile. More particularly, the water-soluble particles generally contain a hydroxy-functional compound that is not polymeric. The term “hydroxy-functional” generally means that the compound contains at least one hydroxyl group, and in certain cases, multiple hydroxyl groups, such as 2 or more, in some embodiments 3 or more, in some embodiments 4 to 20, and in some embodiments, from 5 to 16 hydroxyl groups. The term “non-polymeric” likewise generally means that the compound does not contain a significant number of repeating units, such as no more than 10 repeating units, in some embodiments no or more than 5 repeating units, in some embodiments no more than 3 repeating units, and in some embodiments, no more than 2 repeating units. In some cases, such a compound lacks any repeating units. Such non-polymeric compounds thus a relatively low molecular weight, such as from about 1 to about 650 grams per mole, in some embodiments from about 5 to about 600 grams per mole, in some embodiments from about 10 to about 550 grams per mole, in some embodiments from about 50 to about 500 grams per mole, in some embodiments from about 80 to about 450 grams per mole, and in some embodiments, from about 100 to about 400 grams per mole. Particularly suitable non-polymeric, hydroxy-functional compounds that may be employed in the present disclosure include, for instance, saccharides and derivatives thereof, such as monosaccharides (e.g., dextrose, fructose, galactose, ribose, deoxyribose, etc.); disaccharides (e.g., sucrose, lactose, maltose, etc.); sugar alcohols (e.g., xylitol, sorbitol, mannitol, maltitol, erythritol, galactitol, isomalt, inositol, lactitol, etc.); and so forth, as well as combinations thereof. If utilized, the water-soluble particles typically constitute from about 1 wt.% to about 50 wt.%, in some embodiments from about 2 wt.% to about 45 wt.%, in some embodiments from about 4 wt.% to about 40 wt.%, and in some embodiments, from about 5 wt.% to about 30 wt.% of a release structure layer.

One or more nonionic, anionic, and/or amphoteric surfactants may also be employed to help create a uniform dispersion of materials in the release structure. When employed, such surfactant(s) typically constitute from about 0.05 wt.% to about 8 wt.%, and in some embodiments, from about 0.1 wt.% to about 6 wt.%, and in some embodiments, from about 0.5 wt.% to about 3 wt.% of a release structure layer. Nonionic surfactants, which typically have a hydrophobic base (e.g., long chain alkyl group or an alkylated aryl group) and a hydrophilic chain (e.g., chain containing ethoxy and/or propoxy moieties), are particularly suitable. Some suitable nonionic surfactants that may be used include, but are not limited to, ethoxylated alkylphenols, ethoxylated and propoxylated fatty alcohols, polyethylene glycol ethers of methyl glucose, polyethylene glycol ethers of sorbitol, ethylene oxide-propylene oxide block copolymers, ethoxylated esters of fatty (C₈-C₁₈) acids, condensation products of ethylene oxide with long chain amines or amides, condensation products of ethylene oxide with alcohols, fatty acid esters, monoglyceride or diglycerides of long chain alcohols, and mixtures thereof. Particularly suitable nonionic surfactants may include ethylene oxide condensates of fatty alcohols, polyoxyethylene ethers of fatty acids, polyoxyethylene sorbitan fatty acid esters, and sorbitan fatty acid esters, etc. The fatty components used to form such emulsifiers may be saturated or unsaturated, substituted or unsubstituted, and may contain from 6 to 22 carbon atoms, in some embodiments from 8 to 18 carbon atoms, and in some embodiments, from 12 to 14 carbon atoms. Sorbitan fatty acid esters (e.g., monoesters, diester, triesters, etc.) that have been modified with polyoxyethylene are one particularly useful group of nonionic surfactants. These materials are typically prepared through the addition of ethylene oxide to a 1 ,4-sorbitan ester. The addition of polyoxyethylene converts the lipophilic sorbitan ester surfactant to a hydrophilic surfactant that is generally soluble or dispersible in water. Such materials are commercially available under the designation TWEEN® (e.g., TWEEN® 80, or polyethylene (20) sorbitan monooleate).

Regardless of the particular components employed, the release structure may be formed through a variety of known techniques, such as by hot-melt extrusion, injection molding, solvent casting, dip coating, spray coating, microextrusion, coacervation, compression molding (e.g., vacuum compression molding), etc. In one embodiment, a hot-melt extrusion technique may be employed. Hot-melt extrusion is generally a solvent-free process in which the components of the polymeric matrix (e.g., hydrophobic polymer, hydrophilic compound(s), optional excipients, etc.) may be melt blended and optionally shaped in a continuous manufacturing process to enable consistent output quality at high throughput rates. This technique is particularly well suited to various types of hydrophobic polymers, such as olefin copolymers. Namely, such copolymers typically exhibit a relatively high degree of long-chain branching with a broad molecular weight distribution. This combination of traits can lead to shear thinning of the copolymer during the extrusion process, which help facilitates hot-melt extrusion. Furthermore, polar comonomer units (e.g., vinyl acetate) can serve as an “internal” plasticizer by inhibiting crystallization of the olefin chain segments. This may lead to a lower melting point of the olefin copolymer, which improves the overall flexibility of the resulting material and enhances its ability to be formed into devices of a wide variety of shapes and sizes.

During a hot-melt extrusion process, melt blending may occur at a temperature range of from about 20° C. to about 200° C., in some embodiments, from about 30° C. to about 150° C., in some embodiments from about 40° C. to about 100° C., and in some embodiments, in some embodiments from about 100° C. to about 120° C., to form a polymer composition. Any of a variety of melt blending techniques may generally be employed. For example, the components may be supplied separately or in combination to an extruder that includes at least one screw rotatably mounted and received within a barrel (e.g., cylindrical barrel). The extruder may be a single screw or twin screw extruder. For example, one embodiment of a single screw extruder may contain a housing or barrel and a screw rotatably driven on one end by a suitable drive (typically including a motor and gearbox). If desired, a twin-screw extruder may be employed that contains two separate screws. The configuration of the screw is not particularly critical and it may contain any number and/or orientation of threads and channels as is known in the art. For example, the screw typically contains a thread that forms a generally helical channel radially extending around a core of the screw. A feed section and melt section may be defined along the length of the screw. The feed section is the input portion of the barrel where the olefin copolymer(s) and/or drug compound(s) are added. The melt section is the phase change section in which the copolymer is changed from a solid to a liquid-like state. While there is no precisely defined delineation of these sections when the extruder is manufactured, it is well within the ordinary skill of those in this art to reliably identify the feed section and the melt section in which phase change from solid to liquid is occurring. Although not necessarily required, the extruder may also have a mixing section that is located adjacent to the output end of the barrel and downstream from the melting section. If desired, one or more distributive and/or dispersive mixing elements may be employed within the mixing and/or melting sections of the extruder. Suitable distributive mixers for single screw extruders may include, for instance, Saxon, Dulmage, Cavity Transfer mixers, etc. Likewise, suitable dispersive mixers may include Blister ring, Leroy/Maddock, CRD mixers, etc. As is well known in the art, the mixing may be further improved by using pins in the barrel that create a folding and reorientation of the polymer melt, such as those used in Buss Kneader extruders, Cavity Transfer mixers, and Vortex Intermeshing Pin mixers.

If desired, the ratio of the length (“L”) to diameter (“D”) of the screw may be selected to achieve an optimum balance between throughput and blending of the components. The L/D value may, for instance, range from about 10 to about 50, in some embodiments from about 15 to about 45, and in some embodiments from about 20 to about 40. The length of the screw may, for instance, range from about 0.1 to about 5 meters, in some embodiments from about 0.4 to about 4 meters, and in some embodiments, from about 0.5 to about 2 meters. The diameter of the screw may likewise be from about 5 to about 150 millimeters, in some embodiments from about 10 to about 120 millimeters, and in some embodiments, from about 20 to about 80 millimeters. In addition to the length and diameter, other aspects of the extruder may also be selected to help achieve the desired degree of blending. For example, the speed of the screw may be selected to achieve the desired residence time, shear rate, melt processing temperature, etc. For example, the screw speed may range from about 10 to about 800 revolutions per minute (“rpm”), in some embodiments from about 20 to about 500 rpm, and in some embodiments, from about 30 to about 400 rpm. The apparent shear rate during melt blending may also range from about 100 seconds⁻¹ to about 10,000 seconds⁻¹, in some embodiments from about 500 seconds⁻¹ to about 5000 seconds⁻¹, and in some embodiments, from about 800 seconds⁻¹ to about 1200 seconds⁻¹. The apparent shear rate is equal to 4Q/πR³, where Q is the volumetric flow rate (“m³/s”) of the polymer melt and R is the radius (“m”) of the die (e.g., extruder die) through which the melted polymer flows.

Once melt blended together, the resulting polymer composition may be in the form of pellets, sheets, fibers, filaments, etc., which may be shaped into the desired shape (e.g., a cylinder, a sac, etc.) using a variety of known techniques, such as injection molding, compression molding, nanomolding, overmolding, blow molding, three-dimensional printing, etc. Injection molding may, for example, occur in two main phases - i.e., an injection phase and holding phase. During the injection phase, a mold cavity is filled with the molten polymer composition. The holding phase is initiated after completion of the injection phase in which the holding pressure is controlled to pack additional material into the cavity and compensate for volumetric shrinkage that occurs during cooling. After the shot has built, it can then be cooled. Once cooling is complete, the molding cycle is completed when the mold opens and the part is ejected, such as with the assistance of ejector pins within the mold. Any suitable injection molding equipment may generally be employed in the present disclosure. In one embodiment, an injection molding apparatus may be employed that includes a first mold base and a second mold base, which together define a mold cavity having the shape of the reservoir. The molding apparatus includes a resin flow path that extends from an outer exterior surface of the first mold half through a sprue to a mold cavity. The polymer composition may be supplied to the resin flow path using a variety of techniques. For example, the composition may be supplied (e.g., in the form of pellets) to a feed hopper attached to an extruder barrel that contains a rotating screw (not shown). As the screw rotates, the pellets are moved forward and undergo pressure and friction, which generates heat to melt the pellets. A cooling mechanism may also be provided to solidify the resin into the desired shape of the core (e.g., disc, rod, etc.) within the mold cavity. For instance, the mold bases may include one or more cooling lines through which a cooling medium flows to impart the desired mold temperature to the surface of the mold bases for solidifying the molten material. The mold temperature (e.g., temperature of a surface of the mold) may range from about 30° C. to about 120° C., in some embodiments from about 60° C. to about 110° C., and in some embodiments, from about 30° C. to about 60° C.

Compression molding (e.g., vacuum compression molding) may also be employed. In such a method, a layer of the device may be formed by heating and compressing the polymer compression into the desired shape while under vacuum. More particularly, the process may include forming the polymer composition into a precursor that fits within a chamber of a compression mold, heating the precursor, and compression molding the precursor into the desired layer while the precursor is heated. The polymer composition may be formed into a precursor through various techniques, such as by dry power mixing, extrusion, etc. The temperature during compression may range from about 50° C. to about 120° C., in some embodiments from about 60° C. to about 110° C., and in some embodiments, from about 70° C. to about 90° C. A vacuum source may also apply a negative pressure to the precursor during molding to help ensure that it retains a precise shape. Examples of such compression molding techniques are described, for instance, in U.S. Pat. No. 10,625,444 to Treffer, et al.

### II. Septum

As indicated above, an implantable device can include a septum for refilling the device following implantation. The septum may include a “self-sealing” material that has sufficient properties to allow it to close when a hole is formed by a needle therein. For example, the material used to form the septum may be resilient and can exhibit some elasticity, but is generally firm enough to enable detection of the device beneath the skin by palpation. In other words, the material may be flexible and possess a sufficient degree of hardness without excessive stiffness so as to be self-sealing at thickness of about 1 millimeter or greater. In one embodiment, the septum can include a medical grade silicone polymer. In alternative embodiments, the septum can include other medical grade elastomers or rubbers as are known in the art as well as combinations of materials. Suitable self-sealing materials can include, without limitation, nitrile rubbers, styrene block copolymers, thermoplastic or thermoset polyurethanes, or combinations thereof. In some embodiments, a self-sealing material can exhibit a relatively low hardness, such as a Shore A hardness of about 40 or less, in some embodiments from about 10 to about 30, and in some embodiments, from about 10 to about 20 as determined according to ASTM D2240-15 (2021). Silicone elastomers, such as a dimethyl or dimethyl-diphenyl silicone elastomer, may be used in forming a single-layer or multi-layer septum.

In certain embodiments as illustrated in FIG. 3 and FIG. 4, the septum 121 can be secured to a surrounding clamp or fastener in the form of an encircling retainer ring 130 that can exert radial pressure on the septum 121 and enhance the self-sealing characteristics of the material. The effectiveness of the septum 121 can be improved by the radial compressive force applied to the septum 121 by the retainer ring 130. In general, the greater the radial compressive force applied to septum 121, the higher the self-sealing effectiveness of the septum 121. A retainer ring can be formed of any biocompatible and implantable material capable of exerting desired compressive strength, such as stainless steel, titanium, or the like. In such an embodiment, the septum 121 can include features to allow securement. Such features may include tabs, ridges, recesses, or any other suitable securement features known in the art. In some embodiments, securement between a septum 121 and a retainer ring 130 can be through utilization of a biocompatible adhesive, ultrasonic welding, or combinations of materials and securement methodologies. In some embodiments, the release structure polymer layer 120 can be secured to the septum 121 in conjunction with the clamp or retainer ring 130. By way of example, a clamp (e.g., a retainer ring 130), can be used to compress and secure the release structure polymer 120 against the septum 121 as well as to exert a radial pressure on the septum 121.

The septum 121 may also include a single layer of material or multiple layers of materials placed adjacent to each other. FIG. 5 illustrates a cross-sectional view of one embodiment of a septum 221 that includes multiple layers of material. For instance, a septum 221 can include an interior layer 231 such as an intermediate silicone gel layer encased between two self-sealing elastomer layers 230, 233. A silicone gel layer 231 can aid the septum 221 to seal a hole formed by a penetrating needle and prevent uncontrolled release of fluid from the reservoir.

A septum 121, 221 can optionally include additional layers, such as a mesh layer 235 as in FIG. 5 that may be integrated, or embedded, within a polymeric layer, e.g., elastomer layers 230. A mesh layer 235 can enhance the self-sealing characteristics of a polymeric layer, and can add strength to the septum, allowing the device to maintain desired shape upon manipulation, insertion of needles, and contact with other elements of the surrounding environment (e.g., other parts of the interior of the patient’s body, or forces exerted from outside the patient’s body). In some embodiments, a mesh layer 235 may act in combination with a gel layer 231 to enhance the self-sealing characteristics of a septum 221. For example, after a void is created in the septum 221 by a needle, the gel layer 231 can prevent fluid from having a direct path across the septum 221 and the mesh layer 235 can enhance this property of the gel layer 231 by physically constraining the gel from expansion under pressure exerted by the fluid within the reservoir. When present, material used in forming a mesh layer 235 may include, without limitation, polyethylene terephthalate, polypropylene, polyamide, polyethylene, and combinations thereof as well as any other material capable of producing an equivalent structure.

### III. Backing Layer

As noted above, the device may also include a backing layer 22, 122, extending across portion of the fluid reservoir 40, 140 opposite the septum 21, 121. The backing layer 22, 122 may be positioned inside the release structure polymer 20 (as shown in FIG. 1 and FIG. 2) or may form an external surface of the device (as shown in FIG. 3 and FIG. 4). The backing layer 22, 122 may be bonded to a portion of the polymer release structure 20, 120 through utilization of an adhesive, ultrasonic welding, etc. or may be attached to the polymer release structure 20, 120 through utilization of an attachment mechanism such as hooks, tabs, etc., or some combination thereof. The backing layer 22, 122 may have a disc shape, and may provide structure to one end of a device, for instance in those embodiments in which the release structure polymer 20, 120 is flexible.

Typically, the backing layer is formed from a “puncture-resistant” material to avoid over-penetration of the syringe needle when refilling the reservoir. The puncture-resistant material may deform when contacted by a needle, but the energy required to pass through the backing layer may be great. A person refilling the reservoir by use of the needle will notice the increased resistance and realize the tip of the needle has contacted the side of the reservoir opposite the septum. Examples of suitable puncture-resistant materials may include, for instance, polymeric materials, metals, ceramics, or combinations thereof. Suitable polymeric materials may likewise include polyolefins, polyimides, thermoplastic or thermoset polyurethanes, silicones, acrylonitrile butadiene styrenes, epoxies, rubbers, polyethylene terephthalates, polycarbonates, polyisoprenes, polysulfones, fluoropolymers (e.g., polytetrafluoroethylene), etc. In some embodiments, the backing layer 22, 122 may include a polymeric material that is the form of fibers, such as carbon fibers, glass fibers, quartz fibers, polyester fibers, aramid fibers (e.g., KEVLAR®), etc. The fibers may be in the form of a textile material (e.g., woven fabric, mesh, mat, nonwoven web, etc.) that is formed from such fibers. A metal can be a biocompatible and implantable metal such as, for example, stainless steel, aluminum, titanium, or other metal. The metal can include a metal mesh material having mesh dimensions that are small enough such that a needle (e.g., a 24 gauge needle) cannot pass through it.

In certain embodiments, additional portions (e.g., sidewalls) of the reservoir can also be formed from the same materials as described with regard to the backing layer. For example, the reservoir, excluding the septum, can be formed from with any of the “puncture-resistant” materials as disclosed hereinabove. In a certain embodiment, the reservoir and backing layer can be formed from the same metal material, such as a metal mesh material as described.

In some embodiments, a component of the device, e.g., the backing layer, can include a contrast agent, e.g., barium sulfate, as may be utilized to confirm proper subdermal location of the device following implantation.

### IV. Drug Compound

The drug compound can include any active agent and can be capable of prohibiting and/or treating a condition, disease, and/or cosmetic state a patient. The drug compound may be prophylactically, therapeutically, and/or cosmetically active, either systemically or locally. The dosage level of the drug compound delivered will vary depending on the particular compound employed and the time period for which it is intended to be released. The dosage level is generally high enough to provide a therapeutically effective amount of the drug compound to render a desired therapeutic outcome, e.g., a level or amount effective to reduce or alleviate symptoms of the condition for which it is administered. The exact amount necessary will vary, depending on the subject being treated, the age and general condition of the subject to which the drug compound is to be delivered, the capacity of the subject’s immune system, the degree of effect desired, the severity of the condition being treated, the particular compound selected and mode of administration of the composition, among other factors. An appropriate effective amount can be readily determined by one of skill in the art. For example, an effective amount will typically range from about 5 µg to about 200 mg, in some embodiments from about 5 µg to about 100 mg per day, and in some embodiments, from about 10 µg to about 1 mg of the drug compound delivered per day.

The drug compound can be either naturally occurring or man-made by any method known in the art. Typically, it is also desired that the drug compound is stable at high temperatures so that it can retain desired activity during formation, transport, storage and following insertion into a device reservoir prior to delivery. For example, the drug compound typically remains stable at temperatures of from about 20° C. to about 100° C., in some embodiments from about 30° C. to about 80° C., and in some embodiments, from about 50° C. to about 70° C.

### A. Bisphosphonates

In certain embodiments, the drug compound can include one or more bisphosphonates. Bisphosphonates generally refer to a class of drug compounds that slow down or prevent bone loss. Specifically, bisphosphonates inhibit osteoclasts, which are responsible for breaking down and reabsorbing minerals such as calcium from bone via a process known as bone resorption. Bisphosphonates generally allow osteoblasts to work more effectively, thereby improving bone mass. Bisphosphonates are used in the treatment of osteoporosis, Paget’s disease of bone, and may also be used to lower calcium levels in cancer patients.

The bisphosphonate class of drugs is based on the phosphate-oxygen-phosphate bond (P—O—P) of pyrophosphate (a widely distributed, natural human metabolite that has a strong affinity for bone). Structurally, bisphosphonates are chemically stable derivatives of inorganic pyrophosphate (PPi), a naturally occurring compound in which two phosphate groups are linked by esterification. Replacing the oxygen with a carbon atom (P—C—P) produces a group of bone-selective drugs that cannot be metabolized by the normal enzymes that break down pyrophosphates. The core structure of bisphosphonates differs only slightly from PPi in that bisphosphonates contain a central nonhydrolyzable carbon; the phosphate groups flanking this central carbon are maintained. Nearly all bisphosphonates in current clinical use also have a hydroxyl group attached to the central carbon (termed the R1 position). The flanking phosphate groups provide bisphosphonates with a strong affinity for hydroxyapatite crystals in bone (and are also seen in PPi), whereas the hydroxyl motif further increases a bisphosphonate’s ability to bind calcium. Collectively, the phosphate and hydroxyl groups create a tertiary rather than a binary interaction between the bisphosphonate and the bone matrix, giving bisphosphonates their specificity for bone.

Exemplary bisphosphonates include, but are not limited to, zoledronic acid, risedronate, alendronate, ibandronate, cimadronate, clodronate, tiludronate, minodronate, etidronate, ibandronate, piridronate, pamidronate, 1-fluoro-2-(imidazo-[1 ,2-α]pyridine-3-yl)-ethyl-bisphosphonic acid, and functional analogues thereof. Bisphosphonate compounds can include first-, second-, and third-generation bisphosphonates. For example, early non-nitrogen containing bisphosphonates, including, etidronate, clodronate, and tiludronate, are considered first-generation bisphosphonates. Second- and third-generation bisphosphonates include alendronate, risedronate, ibandronate, pamidronate, and zoledronate (i.e., zoledronic acid). Such second- and third-generation bisphosphonates have nitrogen containing R² side chains. The mechanism by which nitrogen-containing bisphosphonates promote osteoclast apoptosis is distinct from that of the non-nitrogen-containing bisphosphonates. For example, nitrogen-containing bisphosphonates bind to and inhibit the activity of farnesyl pyrophosphate synthase, a key regulatory enzyme in the mevalonic acid pathway critical to the production of cholesterol, other sterols, and isoprenoid lipids. As such, the posttranslational modification (isoprenylation) of proteins (including the small guanosine triphosphate-binding proteins Rab, Rac, and Rho, which play central roles in the regulation of core osteoclast cellular activities including stress fiber assembly, membrane ruffling, and survival) is inhibited, ultimately leading to osteoclast apoptosis.

Salts, esters and/or isomers of bisphosphonates are all meant to be encompassed in the scope of the present disclosure and shall be understood to fall under the term “bisphosphonate”.

### B. Corticosteroids

Drug compounds can also include one or more corticosteroids, including glucocorticoids. Glucocorticoids are defined as a subgroup of corticosteroids. Glucocorticoids, sometimes also named glucocorticosteroids, are a class of steroid hormones that bind to the glucocorticoid receptor and are part of the feedback mechanism of the immune system that turns down immune activity, (e.g., inflammation). In medicine they are used to treat diseases that are caused by an overactive immune system, such as allergies, asthma, autoimmune diseases and sepsis. They also interfere with some of the abnormal mechanisms in cancer cells, so that they are also used to treat cancer. Upon binding the glucocorticoid receptor, the activated glucocorticoid receptor complex up-regulates the expression of anti-inflammatory proteins in the nucleus by a process known as transactivation and represses the expression of pro-inflammatory proteins in the cytosol by attenuating actions on gene induction (via NF-_{K}B, AP1, jun-jun-homoclimers etc.).

Suitable examples of glucocorticoids include hydrocortisone, cortisone acetate, cortisone/cortisol, fluorocortolon, prednisone, prednisolone, methylprednisolone, triamcinolone, dexamethasone, betamethasone, paramethasone. Glucocorticoid polymorphs, isomers, hydrates, solvates, or derivatives thereof are all meant to be encompassed in the scope of the present disclosure and shall be understood to fall under the term “glucocorticoid”.

### C. Selective Estrogen Receptor Modulators (SERMs)

Drug compounds can also include SERMs. SERMs are agents that bind to estrogen receptors but that have the ability to act either as agonists or antagonists in different tissues. For example, in certain SERMs act as agonists on the bone and uterus estrogen receptors and act as antagonists on the breast estrogen receptors. Growth of certain forms of cancers (e.g., breast cancers) may be dependent on estrogen. Accordingly, selective SERMS that act as antagonists on breast tissue are used in the treatment of breast cancer. Additionally, SERMs can be useful in preventing post-menopausal osteoporosis and certain metastatic breast cancers. SERMs are small ligands of the estrogen receptor that are capable of inducing a wide variety of conformational changes in the receptor and thereby eliciting a variety of distinct biological profiles. SERMs not only affect the growth of breast cancer tissue but also influence other physiological processes.

SERMs modulate the proliferation of uterine tissue, skeletal bone density, and cardiovascular health, including plasma cholesterol levels. In general, estrogen stimulates breast and endometrial tissue proliferation, enhances bone density, and lowers plasma cholesterol. Many SERMs are bifunctional in that they antagonize some of these functions while stimulating others. For example, tamoxifen, which is a partial agonist/antagonist at the estrogen receptor inhibits estrogen-induced breast cancer cell proliferation but stimulates endometrial tissue growth and prevents bone loss.

Suitable SERMs include ospemifene, raloxifene, tamoxifene, toremifene, lasofoxifene, bazedoxifene, clomiphene citrate, ormeloxifenem, tibolone, idoxifene, or combinations thereof. SERM polymorphs, isomers, hydrates, solvates, or derivatives thereof are all meant to be encompassed in the scope of the present disclosure and shall be understood to fall under the term “SERM”. Raloxifene and tamoxifene are some of the most commonly prescribed and utilized SERMs.

Raloxifene is an estrogen agonist/antagonist, which belongs to the benzothiophene class of compounds. Raloxifene is represented by structural formula (1).

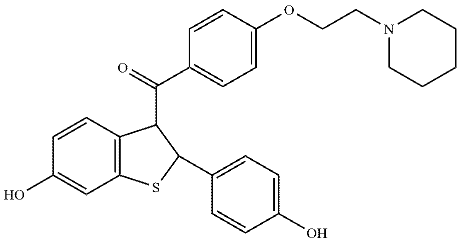 [SMILES: O=C(c1ccc(OCCN2CCCCC2)cc1)C1c2ccc(O)cc2SC1c1ccc(O)cc1]

A chemical name for raloxifene hydrochloride is methanone, [6-hydroxy-2-(4-hydroxyphenyl)benzo[b]thiene-3-yl]-[4-[2-(1-piperidinyl)ethoxy]phenyl]-, hydrochloride. Raloxifene hydrochloride has the empirical formula C₂₈H₂₇NO₄S.HCl, corresponding to a molecular weight of 510.05. Raloxifene hydrochloride is an off-white to pale yellow solid that is very slightly soluble in water, the water solubility being approximately 0.3 g/ml at 25° C., and significantly lower in simulated gastric fluid (SGF) USP (0.003 mg/ml) and simulated intestinal fluid (SIF) USP (0.002 mg/ml), at 37° C. Raloxifene and its derivatives as anti-estrogenic or anti-androgenic compounds are disclosed in U.S. Pat. No. 4,418,068.

Tamoxifen is the trans-isomer of a triphenylethylene derivative. The chemical name is (Z)2-[4-(1,2- diphenyl-1-butenyl)phenoxy]- N,N-dimethylethanamine 2-hydroxy-1,2,3- propanetricarboxylate (1:1). The structural formula, empirical formula, and molecular weight are as follows:

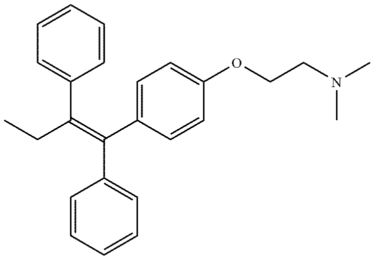 [SMILES: CC/C(=C(\c1ccccc1)c1ccc(OCCN(C)C)cc1)c1ccccc1]

The empirical formula of tamoxifene is C₃₂H₃₇NO₈ and it has a molecular weight of 563.62 Tamoxifen citrate has a pKaʹ of 8.85. The equilibrium solubility in water at 37° C. is 0.5 mg/mL, and is 0.2 mg/mL in 0.02 N HCl at 37° C.

### D. Aromatase Inhibitors

Drug compounds can also include one or more aromatase inhibitors. Aromatase inhibitors refer to a class of agents that are capable of stopping the production of estrogen in post-menopausal women. Aromatase inhibitors work by blocking the enzyme aromatase, which functions to inhibit the conversion of testosterone and/or androgen into estradiol in the body. Accordingly, the reduction in the action of aromatase reduces the amount of estrogen in the body, therefore less estrogen is available to stimulate the growth of hormone-receptor-positive breast cancer cells. Further, aromatase inhibitors do not stop the ovaries from making estrogen, therefore, they are more commonly used to treat postmenopausal women. Aromatase inhibitors are known to cause heart problems and bone loss (e.g., osteoporosis).

Suitable examples of aromatase inhibitors include: exemestane, atamestane, formestane, fadrozole, letrozole, pentrozole, anastrozole, vorozole, or combinations thereof. In another embodiment, the aromatase inhibitor can include non-selective aromatase inhibitors such as Aminoglutethimide and Testolactone (Teslac). In yet another embodiment, aromatase inhibitors may include any other selective or non-selective chemical known to people skilled in the art that inhibits the enzyme aromatase and may prevent estrogen from being formed from its metabolic precursors. Aromatase inhibitor polymorphs, isomers, hydrates, solvates, or derivatives thereof are all meant to be encompassed in the scope of the present disclosure and shall be understood to fall under the term “aromatase inhibitor”.

### E. Antipsychotics

Drug compounds can also include one or more antipsychotics. Antipsychotics generally refer to a class of drug compounds primarily used to manage and treat psychosis, such as schizophrenia. Antipsychotics are also used to treat bipolar disorder and major depressive disorder. Specifically, typical and some atypical antipsychotics are dopamine antagonist and act to impede dopamine in the brain. Further, atypical antipsychotics also influence serotonin.

Exemplary antipsychotics include both typical and atypical antipsychotics. Atypical antipsychotics that can be used herein include, but are not limited to, aripiprazole, clozapine, ziprasidone, paliperidone, risperidone, quetiapine, olanzapine, asenapine, iloperidone, lurasidone, brexpiprazole, cariprazine, and lumateperone. Salts, esters and/or isomers of antipsychotics are all meant to be encompassed in the scope of the present disclosure and shall be understood to fall under the term “antipsychotic”.

In certain embodiments, the drug compound includes risperidone. Risperidone is an atypical antipsychotic and is indicated for the treatment of schizophrenia, irritability associated with autistic disorder, and as monotherapy or adjunctive therapy with lithium or valproate for the treatment of acute manic or mixed episodes associated with Bipolar 1 Disorder. Risperidone belongs to the chemical class of benzisoxazole derivatives. Risperidone has a molecular weight of 410.49 and a molecular formula of C₂₃H₂₇FN₄O₂. The structural formula of risperidone is shown below.

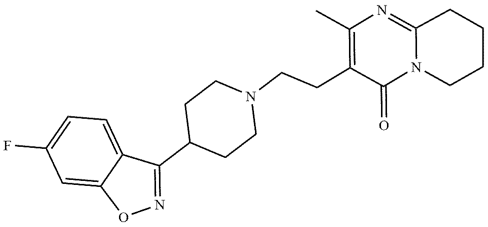 [SMILES: Cc1nc2n(c(=O)c1CCN1CCC(c3noc4cc(F)ccc34)CC1)CCCC2]

Risperidone is a monoaminergic antagonist with high affinity for the serotonin Type 2 (5HT₂), dopamine Type 2 (D₂), α1 and α2 adrenergic, and H₁ histaminergic receptors. Risperidone also shows low to moderate affinity for the serotonin 5HT_{1C}, 5HT_{1D}, 5HT_{1A} receptors and weak affinity for the dopamine D₁ and haloperidol-sensitive sigma site. Risperidone generally shows no affinity for cholinergic muscarinic or β₁ and β₂ adrenergic receptors.

### F. Nucleic Acids

The drug compounds can include nucleic acids, such as naked nucleic acids or encapsulated nucleic acids. As used herein, the term “nucleic acid” generally refers to a compound comprising a nucleobase and an acidic moiety, e.g., a nucleoside, nucleotide, polynucleotide, or a combination thereof. A “nucleoside” generally refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” generally refers to a nucleoside including a phosphate group. Modified nucleotides may by synthesized by any useful method, such as, for example, chemically, enzymatically, or recombinantly, to include one or more modified or non-natural nucleosides. Polynucleotides may comprise a region or regions of linked nucleosides. Such regions may have variable backbone linkages. The linkages may be standard phosphdioester linkages, in which case the polynucleotides would comprise regions of nucleotides. For example, polynucleotides may contain three or more nucleotides are linear molecules, in which adjacent nucleotides are linked to each other via a phosphodiester linkage. The term “nucleic acid” also encompasses RNA as well as single and/or double-stranded DNA. More particularly nucleic acids may be or may include, for example, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2ʹ-amino-LNA having a 2ʹ-amino functionalization, and 2ʹ-amino-c-LNA having a 2ʹ-amino functionalization), ethylene nucleic acids (ENA), cyclohexenyl nucleic acids (CeNA) or chimeras or combinations thereof.

Nucleic acids may be naturally occurring, for example, in the context of a genome, a transcript, a mRNA, tRNA, rRNA, siRNA, snRNA, plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Nucleic acids can be purified from natural sources, produced using recombinant expression systems and optionally purified, chemically synthesized, etc. The nucleic acids may also include nucleoside analogs, such as analogs having chemically modified bases or sugars, and backbone modifications. In some embodiments, the nucleic acid is or contains natural nucleosides (e.g., adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadeno sine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2ʹ-fluororibose, ribose, 2ʹ-deoxyribose, arabinose, and hexose); and/or modified phosphate groups (e.g., phosphorothioates and 5ʹ-N-phosphoramidite linkages).

Modified nucleotide base pairing may be employed and encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the modified nucleotide inosine and adenine, cytosine or uracil. Any combination of base/sugar or linker may be incorporated into polynucleotides of the present disclosure.

In certain embodiments, the nucleic acid may be a polynucleotide (e.g., RNA polynucleotides, such as mRNA polynucleotides) in which one or more nucleobases has been modified for therapeutic purposes. In fact, in certain embodiments, a polynucleotide (e.g., RNA polynucleotide, such as mRNA polynucleotide) may be employed that includes a combination of at least two (e.g., 2, 3, 4 or more) of modified nucleobases. For example, suitable modified nucleobases in the polynucleotide may be a modified cytosine, such as 5-methylcytosine, 5-methyl-cytidine (m5C), N4-acetyl-cytidine (ac4C), 5-halo-cytidine (e.g., 5-iodo-cytidine), 5-hydroxymethyl-cytidine (hm5C), 1-methyl-pseudoisocytidine, 2-thio-cytidine (s2C), 2-thio-5-methyl-cytidine, etc.; modified uridine, such as 5-cyano uridine, 4ʹ-thio uridine, pseudouridine (ψ), N1-methylpseudouridine (m1ψ), N1-ethylpseudouridine, 2-thiouridine (s2U), 4ʹ-thiouridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine (mo5U), 5-methoxyuridine, 2ʹ-O-methyl uridine, etc.; modifined guanosine, such as α-thio-guanosine, inosine (I), 1-methyl-inosine (m1I), wyosine (imG), methylwyosine (mimG), 7-deaza-guanosine, 7-cyano-7-deaza-guanosine (preQO), 7-aminomethyl-7-deaza-guanosine (preQ1), 7-methyl-guanosine (m7G), 1-methyl-guanosine (m1G), 8-oxo-guanosine, 7-methyl-8-oxo-guanosine, etc.; modified adenine, such as α-thio-adenosine, 7-deaza-adenine, 1-methyl-adenosine (m1A), 2-methyl-adenine (m2A), N6-methyl-adenosine (m6A), 2,6-diaminopurine, etc.; as well as combinations thereof. In some embodiments, the polynucleotide (e.g., RNA polynucleotide, such as mRNA polynucleotide) includes a combination of at least two (e.g., 2, 3, 4 or more) of the aforementioned modified nucleobases.

In some embodiments, the polynucleotide (e.g., RNA polynucleotide, such as mRNA polynucleotide) may be uniformly modified (e.g., fully modified, modified throughout the entire sequence) for a particular modification. For example, a polynucleotide can be uniformly modified with 5-methyl-cytidine (m5C), meaning that all cytosine residues in the mRNA sequence are replaced with 5-methyl-cytidine (m5C). Similarly, a polynucleotide can be uniformly modified for any type of nucleoside residue present in the sequence by replacement with a modified residue such as any of those set forth above.

In some embodiments, polynucleotides function as messenger RNA (mRNA). “Messenger RNA” (mRNA) generally refers to any polynucleotide that encodes a (at least one) polypeptide (a naturally-occurring, non-naturally-occurring, or modified polymer of amino acids) and can be translated to produce the encoded polypeptide in vitro, in vivo, in situ or ex vivo. The basic components of a mRNA molecule typically include at least one coding region, a 5ʹ untranslated region (UTR), a 3ʹ UTR, a 5ʹ cap and a poly-A tail. Polynucleotides may function as mRNA but can be distinguished from wild-type mRNA in their functional and/or structural design features that serve to overcome existing problems of effective polypeptide expression using nucleic-acid based therapeutics. The mRNA may contain at least one (one or more) ribonucleic acid (RNA) polynucleotide having an open reading frame encoding at least one polypeptide of interest. In some embodiments, a RNA polynucleotide of a mRNA encodes 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9 or 9-10 polypeptides. In some embodiments, a RNA polynucleotide of a mRNA encodes at least 10, 20, 30, 40, 50, 60, 70, 80, 90 or 100 polypeptides. In some embodiments, a RNA polynucleotide of a mRNA encodes at least 100 or at least 200 polypeptides.

In some embodiments, the nucleic acids are therapeutic mRNAs. As used herein, the term “therapeutic mRNA” refers to a mRNA that encodes a therapeutic protein. Therapeutic proteins mediate a variety of effects in a host cell or a subject in order to treat a disease or ameliorate the signs and symptoms of a disease. For example, a therapeutic protein can replace a protein that is deficient or abnormal, augment the function of an endogenous protein, provide a novel function to a cell (e.g., inhibit or activate an endogenous cellular activity, or act as a delivery agent for another therapeutic compound (e.g., an antibody-drug conjugate). Therapeutic mRNA may be useful for the treatment of various diseases and conditions, such as bacterial infections, viral infections, parasitic infections, cell proliferation disorders, genetic disorders, and autoimmune disorders. The mRNA may be designed to encode polypeptides of interest selected from any of several target categories including, but not limited to, biologics, antibodies, vaccines, therapeutic proteins or peptides, cell penetrating peptides, secreted proteins, plasma membrane proteins, cytoplasmic or cytoskeletal proteins, intracellular membrane bound proteins, nuclear proteins, proteins associated with human disease, targeting moieties or those proteins encoded by the human genome for which no therapeutic indication has been identified but which nonetheless have utility in areas of research and discovery.

Particularly suitable therapeutic mRNAs are those that include at least one ribonucleic acid (RNA) polynucleotide having an open reading frame encoding at least one antigenic polypeptide, in which the RNA polynucleotide of the RNA includes at least one chemical modification. The chemical modification may, for instance, be pseudouridine, N1-methylpseudouridine, N1-ethylpseudouridine, 2-thiouridine, 4ʹ-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine,), 5-methoxyuridine, and 2ʹ-O-methyl uridine.

Although by no means required, the particular nature of the nucleic acid may also be selected to help improve its ability to be dispersed within the polymer matrix and delivered to a patient without significant degradation. For instance, it may be desired to co-deliver a conventional RNA (e.g., mRNA) with a self-amplifying RNA. Conventional mRNAs, for instance, generally include an open reading frame for the target antigen, flanked by untranslated regions and with a terminal poly(A) tail. After transfection, they drive transient antigen expression. Self-amplifying mRNAs, on the other hand, are capable of directing their self-replication, through synthesis of the RNA-dependent RNA polymerase complex, generating multiple copies of the antigen-encoding mRNA, and express high levels of the heterologous gene when they are introduced into the cytoplasm of host cells. Circular RNA (circRNA), which is a single-stranded RNA joined head to tail, may also be employed. The target RNA may be circularized, for example, by backsplicing of a non-mammalian exogenous intron or splint ligation of the 5ʹ and 3ʹ ends of a linear RNA. Examples of suitable circRNAs are described, for instance, in U.S. Pat. Publication No. 2019/0345503, which is incorporated herein by reference thereto. Antisense RNA may also be employed, which generally has a base carried on a backbone subunit composed of morpholino backbone groups and in which the backbone groups are linked by inter-subunit linkages (both charged and uncharged) that allow the bases in the compound to hybridize to a target sequence in an RNA by Watson-Crick base pairing, thereby forming an RNA:oligonucleotide heteroduplex within the target sequence. Morpholino oligonucleotides with uncharged backbone linkages, including antisense oligonucleotides, are detailed, for example, in U.S. Pat. Nos. 5,698,685, 5,217,866, 5,142,047, 5,034,506, 5,166,315, 5,185, 444, 5,521,063, and 5,506,337, which are incorporated herein by reference. Other exemplary antisense oligonucleotides are described in U.S. Pat. Nos. 9,464,292, 10,131,910, 10,144,762, and 10,913,947, which are incorporated herein by reference.

In certain cases, the nucleic acid may be an aptamer, such as an RNA aptamer. An RNA aptamer may be any suitable RNA molecule that can be used on its own as a stand-alone molecule, or may be integrated as part of a larger RNA molecule having multiple functions, such as an RNA interference molecule. For example, an RNA aptamer may be located in an exposed region of an shRNA molecule (e.g., the loop region of the shRNA molecule) to allow the shRNA or miRNA molecule to bind a surface receptor on the target cell. After it is internalized, it may then be processed by the RNA interference pathways of the target cell. The nucleic acid that forms the nucleic acid aptamer may include naturally occurring nucleosides, modified nucleosides, naturally occurring nucleosides with hydrocarbon linkers (e.g., an alkylene), and/or or a polyether linker (e.g., a PEG linker) inserted between one or more nucleosides, modified nucleosides with hydrocarbon or PEG linkers inserted between one or more nucleosides, or a combination of thereof. In some embodiments, nucleotides or modified nucleotides of the nucleic acid aptamer can be replaced with a hydrocarbon linker or a polyether linker. Suitable aptamers may be described, for instance, in U.S. Pat. No. 9,464,293, which is incorporated herein by reference thereto.

Protein-fused nucleic acids may also be suitable for use in the present invention. For example, proteins (e.g., antibodies) may be covalently linked to RNA (e.g., mRNA). Such RNA-protein fusions may be synthesized by in vitro or in situ translation of mRNA pools containing a peptide acceptor attached to their 3ʹ ends. In one embodiment, after readthrough of the open reading frame of the message, the ribosome pauses when it reaches the designed pause site, and the acceptor moiety occupies the ribosomal A site and accepts the nascent peptide chain from the peptidyl-tRNA in the P site to generate the RNA-protein fusion. The covalent link between the protein and the RNA (in the form of an amide bond between the 3ʹ end of the mRNA and the C-terminus of the protein that it encodes) allows the genetic information in the protein to be recovered and amplified (e.g., by PCR) following selection by reverse transcription of the RNA. Once the fusion is generated, selection or enrichment is carried out based on the properties of the mRNA-protein fusion, or, alternatively, reverse transcription may be carried out using the mRNA template while it is attached to the protein to avoid the impact of the single-stranded RNA on the selection. Examples of such protein-fused nucleic acids are described, for instance, in U.S. Pat. No. 6,518,018, which is incorporated herein by reference. Ribozymes (e.g., DNAzyme and/or RNAzyme) may also be employed that are conjugated to nucleic acids having a sequence that catalytically cleaves RNA, such as described in U.S. Pat. No. 10,155,946, which is incorporated herein by reference.

Apart from single strand nucleic acids such as described above, various specific types of double strand nucleic acids may also be employed to help improve stability. Circular DNA (cDNA) and plasmid nucleic acids (e.g., pDNA), which are a closed circular form of DNA, may be employed in certain embodiments. Examples of such nucleic acids are described, for instance, in WO 2004/060277 which is incorporated herein by reference. Long double stranded DNA may also be employed. For instance, a scaffolded DNA origami may be employed in which the long single-stranded DNA is folded into a certain shape by annealing the scaffold in the presence of shorter oligonucleotides (“staples”) containing segments or regions of complementary sequences to the scaffold. Examples of such structures are described, for instance, in U.S. Pat. Publication Nos. 2019/0142882 and 2018/0171386, which are incorporated herein by reference.

### G. Antibodies

The drug compounds can include one or more antibodies. As used herein, the term “antibody” generally includes, by way of example, both naturally occurring and non-naturally occurring Abs, monoclonal and polyclonal Abs, chimeric and humanized Abs; human or nonhuman Abs, wholly synthetic Abs, single chain Abs, etc. A nonhuman Ab may be humanized by recombinant methods to reduce its immunogenicity in man. The term “antibody” also includes an antigen-binding fragment or an antigen-binding portion of any of the aforementioned immunoglobulins, and includes a monovalent and a divalent fragment or portion, and a single chain Ab. Particularly suitable antibodies may include monoclonal antibodies (“MAbs”), multispecific (e.g., bispecific) antibodies, or combinations thereof. The term “monoclonal antibody” generally refers to a non-naturally occurring preparation of Ab molecules of single molecular composition, i.e., Ab molecules whose primary sequences are essentially identical, and which exhibits a single binding specificity and affinity for a particular epitope. Multispecific antibodies, on the other hand, can bind simultaneously different antigens (e.g., two antigens). Such antibodies are generally produced by hybridoma, recombinant, transgenic or other techniques known to those skilled in the art. A “human” antibody refers to an Ab having variable regions in which both the framework and CDR regions are derived from human germline immunoglobulin sequences. Furthermore, if the Ab contains a constant region, the constant region also is derived from human germline immunoglobulin sequences. The human Abs may include amino acid residues not encoded by human germline immunoglobulin sequences (e.g., mutations introduced by random or site-specific mutagenesis in vitro or by somatic mutation in vivo). However, the term “human antibody”, as used herein, is not intended to include Abs in which CDR sequences derived from the germline of another mammalian species, such as a mouse, have been grafted onto human framework sequences.

In some embodiments, the antibody (includes a fragment thereof) can neutralize, block, inhibit, abrogate, reduce, and/or interfere with one or more biological activities (e.g., mitogenic, angiogenic and/or vascular permeability) of a proliferating cell. Such antibodies may, for instance, bind to HER2, TNF-α, VEGF-A, α4-integrin, CD20, CD52, CD25, CD11a, EGFR, respiratory syncytial virus (RSV), glycoprotein IIb/IIIa, IgG1, IgE, complement component 5 (C5), B-cell activating factor (BAFF), CD19, CD30, interleukin-1 beta (IL1β), prostate specific membrane antigen (PSMA), CD38, RANKL, GD2, SLAMF7 (CD319), proprotein convertase subtilisin/kexin type 9 (PCSK9), dabigatran, cytotoxic T-lymphocyte-associated protein 4 (CTLA4), interleukin-5 (IL-5), programmed cell death protein (PD-1), VEGFR2 (KDR), protective antigen (PA) of B. anthracis, interleukin-17 (IL-17), interleukin-6 (IL-6), interleukin-6 receptor (IL6R), interleukin-12 (IL-12), interleukin 23 (IL-23), sclerostin (SOST), myostatin (GDF-8), activin receptor-like kinase 1, delta like ligand 4 (DLL4), angiopoietin 3, VEGFR1, selectin, oxidized low-density lipoprotein (oxLDL), platelet-derived growth factor receptor beta, neuropilin 1, Von Willebrand factor (vWF), neural apoptosis-regulated proteinase 1, beta-amyloid, reticulon 4 (RTN4)/Neurite Outgrowth Inhibitor (NOGO-A), nerve growth factor (NGF), LINGO-1, myelin-associated glycoprotein, etc., as well as combinations thereof.

In one particular embodiment, for example, the antibody may be an anti-PD-1 and/or anti-PD-L1 antibody, such as employed as immune checkpoint inhibitors for treating cancer. PD-1 (or Programmed Death-1) refers to an immunoinhibitory receptor belonging to the CD28 family. PD-1 is expressed predominantly on previously activated T cells in vivo, and binds to two ligands, PD-L1 and PD-L2. The term “PD-1” as used herein includes human PD-1 (hPD-1), variants, isoforms, and species homologs of hPD-1, and analogs having at least one common epitope with hPD-1. The complete hPD-1 sequence can be found under GenBank Accession No. U64863. PD-L1 (or Programmed Death Ligand-1) is one of two cell surface glycoprotein ligands for PD-1 (the other being PD-L2) that downregulate T cell activation and cytokine secretion upon binding to PD-1. The term “PD-L1” as used herein includes human PD-L1 (hPD-LI), variants, isoforms, and species homologs of hPD-LI, and analogs having at least one common epitope with hPD-LI. The complete hPD-LI sequence can be found under GenBank Accession No. Q9NZQ7. HuMAbs that bind specifically to PD-1 with high affinity have been described, for instance, in U.S. Pat. Nos. 8,008,449 and 8,779,105. Other anti-PD-1 mAbs have been described in, for example, U.S. Pat. Nos. 6,808,710, 7,488,802, 8,168,757 and 8,354,509, and PCT Publication No. WO 2012/145493. For example, the anti-PD-1 MAb may be nivolumab. Nivolumab (also known as Opdivo®; formerly designated 5C4, BMS-936558, MDX-1106, or ONO-4538) is a fully human IgG4 (S228P) PD-1 immune checkpoint inhibitor Ab that selectively prevents interaction with PD-1 ligands (PD-LI and PD-L2), thereby blocking the down-regulation of antitumor T-cell functions (U.S. Pat. No. 8,008,449). In another embodiment, the anti-PD-1 mAb is pembrolizumab, as well as antigen-binding variants thereof. Pembrolizumab (also known as Keytruda®, lambrolizumab, and MK-3475) is a humanized monoclonal IgG4 antibody directed against human cell surface receptor PD-1 (programmed death- 1 or programmed cell death-1). Pembrolizumab is described, for example, in U.S. Pat. Nos. 8,354,509 and 8,900,587). In other embodiments, the anti-PD-1 MAb is MEDI0608 (formerly AMP-514) as described, for example, in U.S. Pat. No. 8,609,089. Yet other examples of humanized monoclonal antibodies include Pidilizumab (CT-011), BGB-A317, etc., as well as antigen-binding variants thereof.

In another particular embodiment, for example, the antibody may be an anti-CTLA-4 (cytotoxic T-lymphocyte associated protein-4) antibody, such as employed as immune checkpoint inhibitors for treating cancer. CLTA-4 is a protein receptor that downregulates the immune system. The term “CTLA-4” as used herein includes human CTLA-4(hCTLA-4), variants, isoforms, and species homologs of hCTLA-4, and analogs having at least one common epitope with hCTLA-4. Specifically, CLTA-4 is an immunoglobulin cell surface receptor and an inhibitor of T cell activation and primarily expresses naive T cells after activation and FoxP3+ regulatory T cells (Tregs). T cell activation is dependent not only on T cell receptor (TCR) binding with an antigen presented via an Adenomatous polyposis coli (APC), but also in the presence of a costimulatory second signal, typically through binding of CD28 expressed on the T cell to CD80/86 found on the APC. Absences of this secondary signal may lead the T cell to recognize the presented peptide as a “self-antigen” or to develop tolerance to the antigen. CTLA-4 is a competitive homolog for CD28 that has a higher affinity to CD80 (B7-1), and to a lesser extent CD68 (B7-2) compared with CD28, leading to inhibition of T cell stimulation. TCR signaling immediately upregulates cell surface CTLA-4 expression, reaching peak expression at 2 to 3 days after activation, providing a negative feedback loop upon T cell activation. CTLA-4 within intracellular vesicles is also quickly transported to the immunologic synapse following T cell activation. At the immunologic synapse, CTLA-4 is stabilized by CD80/CD86 binding, allowing it to collect and inhibit CD28 binding. Accordingly, inclusion of an anti-CTLA-4 antibody can disrupt the inhibitory mechanism of CTLA-4. Antibodies that bind specifically to CLTA-4 have been described, for instance, in in U.S. Pat. Nos. 6,984,720 and U.S. 10,196,445.

In certain embodiments, the anti-CTLA-4 antibody can be Ipilimumab. Ipilimumab (also known as Yervoy®, designated MDX101) is a fully humanized IgG1 kappa immulgolubulin directed against CTLA-4. Ipilimumab has an approximate molecular weight of 148 kDa. Ipilimumab is produced in mammalian (Chinese hamster ovary) cell culture. Ipilimumab is a negative regulator of T-cell activity. Ipilimumab binds to CTLA-4 and blocks the interaction of CTLA-4 with its ligands, CD80 and CD86. Blockage of CTLA-4 by Ipilimumab has been shown to augment T-cell activation and proliferation, including the activation and proliferation of tumor infiltrating T-effector cells. Inhibition of CLTA-4 signaling can also reduce T-regulatory cell function, which may contribute to a general increase in T cell responsiveness, including the anti-tumor immune response.

Another example of a suitable antibody is an anti-VEGF antibody, which is an antibody or antibody fragment (e.g., Fab or a scFV fragment) that specifically binds to a VEGF receptor. Anti-VEGF antibodies act, for example, by interfering with the binding of VEGF to a cellular receptor, by interfering with vascular endothelial cell activation after VEGF binding to a cellular receptor, and/or by killing cells activated by VEGF. An anti-VEGF antibody will usually not bind to other VEGF homologues (e.g., VEGF-B or VEGF-C) or other growth factors (e.g., PIGF, PDGF or bFGF). Suitable anti-VEGF antibodies may include monoclonal and/or bispecific anti-VEGF antibodies, such as A4.6.1, bevacizumab, ranibizumab, G6, B20, 2C3, and other antibodies such as described in U.S. Pat. Nos. 6,582,959, 6,703,020, 7,060,269, 7,169,901, 7,691,977, and 10,590,193; U.S. Pat. Publication No. 2009/0169556; WO 94/10202; WO 98/45332; WO 96/30046, WO 2019/154776; WO 2010/040508; WO 2011/117329; WO0 2012/131078; WO 2015/083978; WO 2017/197199; and WO 2014/009465, all of which are incorporated herein by reference. For example, the anti-VEGF antibody may be ranibizumab, bevacizumab, , as well as antigen-binding variants thereof. Ranibizumab (molecular weight of 48 kD) is a humanized monoclonal Fab fragment directed against VEGF-A having the light and heavy chain variable domain sequences of Y0317 as described in SEQ ID Nos. 115 and 116 of U.S. Pat. No. 7,060,269. Ranibizumab inhibits endothelial cell proliferation and neovascularisation and may be used for the treatment of neovascular (wet) age-related macular degeneration (AMD), the treatment of visual impairment due to diabetic macular oedema (DME), the treatment of visual impairment due to macular oedema secondary to retinal vein occlusion (branch RVO or central RVO), or treatment of visual impairment due to choroidal neovascularisation (CNV) secondary to pathologic myopia. Bevacizumab (molecular weight of 149 kD) is likewise a full-length, humanized murine monoclonal antibody that recognizes all isoforms of VEGF and which is the parent antibody of ranibizumab. In another embodiment, the anti-VEGF antibody may also be a bispecific antibody that contains a first antigen binding site that binds to human vascular endothelial growth factor (e.g., VEGF-A) and a second antigen binding site that binds to human angiopoietin-2 (ANG-2). One example of such an anti-VEGF antibody is faricimab (molecular weight of 150 kD), which is described in WO 2019/154776 and WO 2014/009465 and is composed of an anti-Ang-2 antigen-binding fragment (Fab), an anti-VEGF-A Fab, and a modified fragment crystallizable region (Fc region).

Yet another suitable antibody is an anti-HER2 antibody. Such antibodies may be full length anti-HER2 antibodies; anti-HER2 antibody fragments having the same biological activity; including amino acid sequence variants and/or glycosylation variants of such antibodies or antibody fragments. Examples of humanized anti-HER2 antibodies include trastuzumab, pertuzumab, and margetuximab, as well as antigen-binding variants thereof. Yet other anti-HER2 antibodies with various properties have been described in Tagliabue et al., Int. J. Cancer, 47:933-937 (1991); McKenzie et al., Oncogene, 4:543-548 (1989); Cancer Res., 51:5361-5369 (1991); Bacus et al., Molecular Carcinogenesis, 3:350-362 (1990); Stancovski et al., PNAS (USA), 88:8691-8695 (1991); Bacus et al., Cancer Research, 52:2580-2589 (1992); Xu et al., Int. J. Cancer, 53:401-408 (1993); WO94/00136; Kasprzyk et al., Cancer Research, 52:2771-2776 (1992); Hancock et al., Cancer Res., 51:4575-4580 (1991); Shawver et al., Cancer Res., 54:1367-1373 (1994); Arteaga et al., Cancer Res., 54:3758-3765 (1994); Harwerth et al., J. Biol. Chem., 267:15160-15167 (1992); U.S. Pat. No. 5,783,186; and Klapper et al., Oncogene, 14:2099-2109 (1997).

Yet another suitable antibody is an anti-cKIT antibody. Such antibodies may be full length anti-cKIT antibodies; anti-cKIT antibody fragments having the same biological activity; including amino acid sequence variants and/or glycosylation variants of such antibodies or antibody fragments. cKIT is a single transmembrane, receptor tyrosine kinase inhibitor that binds the ligand stem cell factor (SCF). SCF induces homodimerization of cKIT, which activates its tyrosine kinase activity and signals through both the PI3-AKT and MAPK pathways as described in Kindblom et al., Am J. Path. 1998 152(5): 1259. Anti-cKIT antibodies with various properties are described in Gadd et al., Leuk. Res. 1985 (9): 1329, Broudy et al., Blood 1992 79(2):338, U.S. Pat. No. 8,552,157, and U.S. Pat. No. 9,540,443.

Another suitable antibody is an anti-4-1BB antibody (anti-CD137). CD137 (4-1BB) is a member of the tumor necrosis receptor (TNF-R) gene family, which includes proteins involved in regulation of cell proliferation, differentiation, and programmed cell death. Suitable anti-4-1BB antibodies include urelumab (BMS-663513), which is a fully human IgG4 monoclonal antibody. Other suitable anti-4-1BB antibodies are described in U.S. Pat. No. 7,288,638, U.S. Pat. No. 7,659,384, U.S. Pat. No. 8,137,667, U.S. Patent. No. 10,875,921, U.S. Pat. No. 11,242,395.

Typically, the antibody is present in the formulation as a naked antibody. However, if desired, the drug compound can include an antibody drug conjugate (ADC). ADCs are a rapidly growing class of targeted therapeutics, represent a promising new approach toward improving both the selectivity and the cytotoxic activity of cancer drugs (e.g., cytotoxic agents). ADCs have three components: (1) a monoclonal antibody conjugated through a (2) linker to a (3) cytotoxin. The cytotoxins are attached to either lysine or cysteine sidechains on the antibody through linkers that react selectively with primary amines on lysine or with sulfhydryl groups on cysteine.

The maximum number of linkers/drugs that can be conjugated depends on the number of reactive amino or sulfhydryl groups that are present on the antibody. A typical antibody contains up to 90 lysines as potential conjugation sites; however, the typical number of cytotoxins per antibody for most ADCs is typically between 2 and 4 due to aggregation of ADCs with higher numbers of cytotoxins. As a result, conventional lysine linked ADCs are heterogeneous mixtures that contain from 0 to 10 cytotoxins per antibody conjugated to different amino groups on the antibody. Antibody cysteines can also be used for conjugation to cytotoxins through linkers that contain maleimides or other thiol specific functional groups. A typical antibody contains 4, or sometimes 5, interchain disulfide bonds (2 between the heavy chains and 2 between heavy and light chains) that covalently bond the heavy and light chains together and contribute to the stability of the antibodies in vivo. These interchain disulfides can be selectively reduced with dithiothreitol, tris(2-carboxyethyl)phosphine, or other mild reducing agents to afford 8 reactive sulfhydryl groups for conjugation. Cysteine linked ADCs are less heterogeneous than lysine linked ADCs because there are fewer potential conjugation sites; however, they also tend to be less stable due to partial loss of the interchain disulfide bonds during conjugation, since current cysteine linkers bond to only one sulfur atom. The typical number of cytotoxins per antibody for cysteine linked ADCs is also 2 to 4. For example, ADCETRIS is a heterogeneous mixture that contains 0 to 8 monomethylauristatin E residues per antibody conjugated through cysteines.

Key factors in the success of an ADC include that the monoclonal antibody is cancer antigen specific, non-immunogenic, low toxicity, and internalized by cancer cells; the cytotoxin is highly potent and is suitable for linker attachment; while the linker may be specific for cysteine (S) or lysine (N) binding, is stable in circulation, may be protease cleavable and/or pH sensitive, and is suitable for attachment to the cytotoxin. When used to treat cancer, for example, the ADC and/or antigen to which the antibody is bound may be internalized by the cell, resulting in increased therapeutic efficacy of the ADC in killing the cancer cell to which it binds.

The ADC can include at least one of the monoclonal antibodies described hereinabove with at least one cytotoxic agent (e.g., a chemotherapeutic agent as described hereinbelow). For instance, the ADC can comprise an anti-CLTA-4 antibody (e.g., ipilimumab) linked to one or more chemotherapeutic agents. In another example, the ADC can include an anti-PD1 antibody (e.g., nivolumab, pembrolizumab, or pidilizumab) linked to one or more chemotherapeutic agents. In another example, the ADC can include an anti-VEGF antibody (e.g., faricimab) linked to one or more chemotherapeutic agents. In another example, the ADC can include an anti-HER2 antibody (e.g., trastuzumab, pertuzumab, and margetuximab) linked to one or more chemotherapeutic agents. In yet another example, the ADC can include an anti-cKIT antibody linked to one or more chemotherapeutic agents. Suitable ADCs including anti-cKIT antibodies are described in PCT Publication No. WO 2014/150937.

The number of molecules of a drug (e.g., chemotherapeutic agent) conjugated per antibody is known as the drug-to-antibody ratio (DAR). The DAR can affect the potency and overall toxicity of the ADC. For instance, if the DAR is too low then the ACD may not be capable of providing therapeutically effective results, while if the DAR is too high, the patient may experience unwanted side effects. Given reaction and processing conditions, the DAR for ADS typically ranges anywhere from 0 to 15, such as from about 0 to 8. Accordingly, the ADCs as disclosed can have a DAR ranging from 0 to 15, such as 1 to 14, such as 2 to 13, such as 3 to 12, such as 4 to 11, such as 5 to 10, such as 6 to 9. In certain embodiments, the ADC has a DAR of 2 to 4.

The DAR for a manufacturing batch of ADC can be determined empirically using spectrophotometric measurements and ADC therapeutic compositions typically contain a mixture of ADC species that differ in drug load. Thus, the DAR for an ADC batch represents the average DAR of the ADC species within the batch. Varying DARs per batch contributes to potency variability. In order to reduce potency variability, the ADCs contained within a batch utilized in the present pharmaceutical formulation can include an average DAR of from about 2 to about 8, such as from about 2 to about 6, such as from about 2 to 4. In a certain example, the average DAR of the ADCs is about 3 to about 5, such as about 4.

Suitable FDA-approved ADCs for use in the pharmaceutical formulation of the present disclosure also include gemtuzumab ozogamicin (Mylotarg™), brentuximab vedotin (Adetris™), trastuzumab ematansine (Kadcyla™), inotuzumab ozogamicin (Besponsa™), moxetumomab pasudotox (Lumoxiti™), polatuzumab vedotin-piiq (Polivy™), enfortumab vedontin (Padcev™), trastuzumab deruxtecan (Enhertu™), sacituzumab govitecan (TrodeIvy™), belantamab mafodotin-blmf (Blenrep™), locastuximab tesirine-lpyl (Zylonta™), tisotumab vedotin-tftv (Tivdak™), and combinations thereof. Other suitable ADCs include mirvetuximab soravtansin (IMGN853), transtuzumab duocarmazine (SYD985), depatuxizumab mafodotin (AGX-414), disitimab vedotin (RC48-ADC), and combinations thereof. Other suitable ADCs include Mirvetuximab soravtansine (IMGN853), Transtuzumab duocarmazine (SYD985), Depatuxizumab mafodotin (ABT-414), Disitimab vedotin (RC48-ADC), and combinations thereof. In other embodiments, the ADC can include an antibody that is linked to other types of molecules such as oligonucleotides, radionuclides, and protein toxins.

Antibodies of the present disclosure can also include radiolabeled antibodies. Such antibodies include a radioactive substance conjugated to the antibody configured to provide radioactivitiy directly to cancer cells. Suitable radiolabeled antibodies include ibritumomab tiuxetan (Zevalin™). Radiolabeled antibodies include any of the antibodies disclosed herein that are conjugated with a radioactive material, such as Yttrium-90. Other radiolabeled antibodies are described in International Patent Application No. WO 2009/0538203 and U.S. Pat. Application No. 7,402,385.

Antibodies of the present disclosure can also include multispecific antibodies, such as bispecific antibodies (BsAbs). Multispecific antibodies refers to any antibody that can bind simultaneously to two or more different antigens, while BsAbs refers to an antibody that can bind simultaneously to two different antigens. One example BsAb is a bispecific T cell engager (BiTE) with one arm targeting CD3 on T cells and the other recognizing target proteins on tumor cells, thereby activating the T cells to kill the tumor cells. In addition to their interaction with T cells, BsABs have also been designed to engage other effector ells, such as natural killer (NK) cells and macrophages for cancer therapy. Suitable BsAbs include blinatumomab (BLINCYTO™) which targets both CD19 and CD3 and catumaxomab (Removab™) which targets human EpCAM and human CD3 receptors. Other suitable BsAbs include AMG 330 (anti-CD3/CD33), AMG 427 (anti-CD3/FLT3), AMG 673 (anti-CD3/CD33), AMG 701 (anti-CD3/BCMA), AMG 160 (anti-CD3/prostate specific membrane antigen (PSMA)), AMG 596 (anti-CD3/epidermal growth factor receptor (EGFR) and AMG 757 (anti-CD3/DLL-3), all developed by Amgen.

Other suitable BsABs or multispecific antibodies can include those currently FDA-approved and/or under clinical trial testing including Blinatumomab/Blincyto/MT103/MEDI-538/AMG103 (clinical trials identifiers NCT01466179 NCT02013167), AFM11 (clinical trials identifier NCT02848911), AMG562 (clinical trials identifier NCT03571828), REGN1979 (clinical trials identifier NCT03888105), Glofitamab/RO7082859 (clinical trials identifier NCT03075696), Plamotamab/XmAb13676 (clinical trials identifier NCT02924402), Mosunetuzumab/RG7828/RO703081 (clinical trials identifier NCT04313608), GEN3013 (clinical trials identifier NCT03625037), AMG673 (clinical trials identifier NCT03224819), AMV-564 (clinical trials identifier NCT03144245), ISB 1342 (clinical trials identifier NCT03309111), JNJ-63709178 (clinical trials identifier NCT02715011), SAR440234 (clinical trials identifier NCT03594955), Vibecotamab/Xmab14045 (clinical trials identifier NCT02730312), AMG420/BI 836909 (clinical trials identifier NCT03836053), CC-93269/EM801 (clinical trials identifier NCT03486067), Teclistamab/JNJ-64007957 (clinical trials identifier NCT04557098), PF-06863135 (clinical trials identifier NCT04649359), REGN5458 (clinical trials identifier NCT03761108), Catumaxomab/removab (clinical trials identifier NCT03070392), RG6194/BTRC4017A (clinical trials identifier NCT03448042), M802 (clinical trials identifier NCT04501770), GBR1302 (clinical trials identifier NCT03983395), Cibisatamab/RG7802/RO6958688 (clinical trials identifier NCT03866239), AMG211 (clinical trials identifier NCT02291614), AMG160 (clinical trials identifier NCT03792841), MOR209/ES414 (clinical trials identifier NCT02262910), Pasotuxizumab/BAY2010112 (clinical trials identifier NCT01723475), REGN5678 (clinical trials identifier NCT03972657), FS120 (clinical trials identifier NCT04648202), PRS-343 (clinical trials identifier NCT03330561), AFM13 (clinical trials identifiers NCT03192202, NCT04101331), AFM24 (clinical trials identifier NCT04259450), GTB-3550, OXS-35504 (clinical trials identifier NCT03214666), MEDI5752 (clinical trials identifier NCT04522323), AK104 (clinical trials identifier NCT04172454), XmAb20717 (clinical trials identifier NCT03517488), MGD019 (clinical trials identifier NCT03761017), MGD013 (clinical trials identifier NCT03219268), RO7121661, RG7769 (clinical trials identifier NCT03708328), KN046 (clinical trials identifiers NCT03872791, NCT04474119, NCT04469725, NCT03733951), FS118 (clinical trials identifier NCT03440437), LY3415244 (clinical trials identifier NCT03752177), IBI318/LY3434172 (clinical trials identifier NCT03875157), IBI315 IBI318/LY3434172 (clinical trials identifier NCT04162327), AK112 (clinical trials identifier NCT04047290), IBI319 (clinical trials identifier NCT04708210), FS222 (clinical trials identifier NCT04740424), MCLA-145 (clinical trials identifier NCT03922204), ATOR 1015 (clinical trials identifier NCT03782467), XmAb23104 (clinical trials identifier NCT03752398), TG-1801/NI-1701 (clinical trials identifier NCT03804996), IMM0306 (clinical trials identifier CTR20192612), IBI322 (clinical trials identifiers NCT04338659, NCT04328831), HX009 (clinical trials identifier NCT04097769), JNJ-61186372/Amivantamab (clinical trials identifier NCT02609776), MCLA-158 (clinical trials identifier NCT03526835), MCLA-128/Zenocutuzumab (clinical trials identifier NCT03321981), KN026 (clinical trials identifier NCT04521179), MBS301 (clinical trials identifier NCT03842085), ZW25 (clinical trials identifier NCT02892123), ZW49 (clinical trials identifier NCT03821233), MM-141 (clinical trials identifier NCT02399137), BI 836880 (clinical trials identifiers NCT03972150, NCT03697304), RO5520985/Vanucizumab (clinical trials identifier NCT02141295), ABT-165/Dilpacimab (clinical trials identifier NCT01946074), OMP-305B83/Navicixizumab (clinical trials identifier NCT03030287), RG7386/RO6874813 (clinical trials identifier NCT02558140), OXS-1550/DT2219ARL (clinical trials identifier NCT02370160), and combinations thereof.

As noted above, the antibodies can include antibody fragments (Fabs). Such FDA-approved Fabs include Ranibizumab (Lucentis™), Abciximab (ReoPro™), Certolizumab pegol (Cimzia™), Idarucizumab (Praxbind™), Digoxin Immune Fab (DigiFab™), crotalidae - polyvalent immune fab (CroFab™), arotalidae - polyvalent immune Fab (Anavip™), centruroides immune F(abʹ)2 (Anascorp™), Brolucizumab (Beovu™), Caplacizumab (Cablivi™), or combinations thereof. Additionally Fabs that can be utilized in the present device also include Copper Cu 64-DOTA-B-Fab (clinical trials identifier NCT02708511), CSR02-Fab-TF (clinical trials identifier NCT04601428), Ranibizumab (clinical trials identifier NCT00540930), Naptumomab estafenatox (clinical trials identifier NCT00420888), IMCgp100 (clinical trials identifier NCT01209676), L19-IL2 (clinical trials identifier NCT02086721), rM28 (clinical trials identifier NCT00204594), D2C7-IT (clinical trials identifier NCT02303678), NM21-1480 (clinical trials identifier NCT04442126), Vicinium (clinical trials identifier NCT02449239), [124 I] PSCA-Minibody (clinical trials identifier NCT02092948), 6B11-OCIK (clinical trials identifier NCT03542669), T84.66 (clinical trials identifier NCT00647153), BCMA VHH CAR-T Cell (clinical trials identifier NCT03664661), CD19/20 bispecific VHH-derived CAR-T Cells (clinical trials identifier NCT03881761), ALX-0651 (clinical trials identifier NCT01374503), αPD1-MSLN-CAR T cells (clinical trials identifiers NCT04489862, NCT04503980), [131I]-SGMIB anti-HER2 VHH1 (clinical trials identifier NCT02683083), 68-Ga NOTA-anti-MMR-VHH2 (clinical trials identifier NCT04168528), 68-GaNOTA-anti-HER2 VHH1 VHH (clinical trials identifiers NCT03924466, NCT03331601), 99mTc-MIRC208 (clinical trials identifier NCT04591652), TAS226 (clinical trials identifier NCT01529307), and combinations thereof.

In embodiments, the antibodies can include antibody cytokine fusion proteins fusion proteins, referred to as immunocytokines. Cytokines constitute a broad and loosely defined class of relatively small proteins that regulate the immune response. The systemic administration of proinflammatory cytokines is often associated with severe off target toxicity, particularly flu-like symptoms, which may limit the dose and prevent the escalation of dosages needed for developing therapeutically effective regimens. Similar to ADCs, utilization of cytokines with antibodies or antibody fragments as vehicles has been used for the targeted delivery of immunomodulatory cytokines (such as interleukin (IL)-2, IL-12, and tumor necrosis factor (TNF) including TNFa and TNFb) to leverage the local tumor microenvironment (TME) and activate anticancer immune responses. Suitable immunocytokines that may be used in accordance with the present disclosure include, but are not limited to, L19IL2 (clinical trials identifier NCT01058538), L19TNFa (clinical trials identifier NCT01253837), F16IL2 (clinical trials identifier NCT01134250), hu14.18-IL2 (clinical trials identifier NCT00003750), huKS-IL2 (EMD 273066) (clinical trials identifier NCT00132522), DI-Leu16-IL2 (anti-CD20-IL2) (clinical trials identifier NCT01374288), NHS-IL12 (clinical trials identifier NCT04303117), NHS-IL2-LT (EMD 521873) (clinical trials identifier NCT00879866), Anti-CEA-IL2v (cergutuzumab amunaleukin) (clinical trials identifier NCT02350673), and combinations thereof.

Antibodies of the present disclosure also include antibody-small interfering RNA (siRNA) conjugates (ARCs). Since antibodies show high specificity toward overexpressed antigens in certain cell types or tissues, antibodies can be utilized to target delivery of siRNA molecules. siRNA molecules can be linked to the antibodies with covalent linkages with lysine or cysteine residues. Accordingly, antibodies utilized herein can include the disclosed antibodies linked with one or more siRNA molecules for treating cancer. Such ARCs can be used to treat a variety of cancers. Utilization of ARCs to treat cancer has had a few drawbacks, namely ARCs may not readily enter the cell due to the negative charge of the appended siRNA, which makes it difficult to overcome the thermodynamic barriers presented by the cell membrane. As such, ARCs can be incorporated into the device of the present disclosure and released within the tumor cells themselves, eliminating thermodynamic barriers at the cell membrane.

### H. Chemotherapeutic Agents

Drug compounds can also include chemotherapeutic agents. Suitable chemotherapeutic agents include, but are not limited to alkylating agents, platinum drugs, antimetabolites, antitumor antibiotics, topoisomerase inhibitors, mitotic inhibitors, corticosteroids, and other miscellaneous chemotherapeutic agents. Alkylating agents directly damage DNA to prevent the cancer cell from reproducing illustrative examples of which include, nitrogen mustards (such as, mechlorethamine (nitrogen mustard), chlorambucil, cyclophosphamide (Cytoxan®), ifosfamide, and melphalan), nitrosoureas (including streptozocin, carmustine (BCNU), and lomustine) , alkyl sulfonates (e.g., busulfan) , triazines (such as, dacarbazine (DTIC) and temozolomide (TEMODAR®), and ethylenimines (e.g., thiotepa and altretamine (hexamethylmelamine)). The platinum drugs are sometimes grouped with alkylating agents because they kill cells in a similar way. Examples of platinum drugs include cisplatin, carboplatin, and oxalaplatin. Antimetabolites interfere with DNA and RNA growth by substituting for the normal building blocks of RNA and DNA. Examples of antimetabolites include, 5-fluorouracil (5-FU), 6-mercaptopurine (6-MP), capecitabine (XELODA®), cladribine, clofarabine, cytarabin (ARA-C®), foxuridine, fludarabine, gemcitabine (GEMZAR®), hydroxyurea, methotrexate, pemetrexed (ALIMTA®), pentostatin, and thioguanine. Anti-tumor antibiotics either break down DNA strands or slow down or stop DNA synthesis, thereby retarding the proliferation of cancer cells. Currently available anti-tumor antibiotics include anthracyclines (such as, daunorubicin, doxorubicin (ADRIAMYCIN®), epirubicin, and idarubicin), actinomycin-D, bleomycin, dactinomycin, mitomycin-C, and plicamycin. Topoisomerase inhibitors interfere with topoisomerases, which help separate the strands of DNA during cell proliferation. Examples of topoisomerase I inhibitors include camptothecin (CPT), irinotecan (CPT-11), and topotecan. Examples of topoisomerase II inhibitors include etoposide (VP-16), mitoxantrone, and teniposide. Mitotic inhibitors can stop mitosis or inhibit the synthesis of proteins involved in cell reproduction. Examples of mitotic inhibitors include, taxanes (such as, paclitaxel (TAXOL®) and docetaxel (TAXOTERE®)), epothilones, (e.g., ixabepilone (IXEMPRA®)), vinca alkaloids (such as, vinblastine, vincristine (ONCOVlN®), and vinorelbine (NAVELBINE®)), and estramustine.

If desired, the drug compound may be provided in the form of a composition, such as a solution, dispersion, suspension, emulsion, etc., as well as a powder that can be reconstituted into a liquid form prior to delivery. Examples of suitable aqueous and nonaqueous carriers, diluents, solvents or vehicles for use in such compositions may include water, ethanol, polyols (e.g., glycerol, propylene glycol, polyethylene glycol and the like), carboxymethylcellulose and suitable mixtures thereof, vegetable oils (e.g., olive oil), and injectable organic esters such as ethyl oleate. Typically, the drug compound will constitute from about 5 wt.% to about 60 wt.%, in some embodiments from about 10 wt.% to about 50 wt.%, and in some embodiments, from about 15 wt.% to about 45 wt.% of the composition, while water and other optional components can constitute from about 40 wt.% to about 95 wt.%, in some embodiments from about 50 wt.% to about 90 wt.%, and in some embodiments, from about 55 wt.% to about 85 wt.% of the composition. The composition may also optionally contain one or more excipients if so desired, such as radiocontrast agents, release modifiers, bulking agents, plasticizers, surfactants, flow aids, colorizing agents (e.g., chlorophyll, methylene blue, etc.), antioxidants, stabilizers, lubricants, other types of antimicrobial agents, preservatives, etc. to enhance properties and processability. When employed, the optional excipient(s) typically constitute from about 0.01 wt.% to about 20 wt.%, and in some embodiments, from about 0.05 wt.% to about 15 wt.%, and in some embodiments, from about 0.1 wt.% to about 10 wt.% of the composition. Antimicrobial agents and/or preservatives may be employed, for instance, to help prevent surface growth and attachment of bacteria, such as metal compounds (e.g., silver, copper, or zinc), metal salts, quaternary ammonium compounds, etc. The composition can contain minor amounts of auxiliary substances, such as wetting or emulsifying agents, pH buffering agents and the like that can enhance the effectiveness of the active ingredient. Proper fluidity may be maintained, for example, by the use of coating materials such as lecithin, by the maintenance of the required particle size in the case of dispersions and by the use of surfactants. The composition also may contain adjuvants such as wetting agents, emulsifying agents and dispersing agents. It also may be desirable to include isotonic agents such as sugars, sodium chloride and the like.

### V. Use of Device

The implantable device may be used in a variety of different ways to treat prohibit and/or treat a condition, disease, or cosmetic state in a patient. The term “implantable device” as used herein, is intended to cover a variety methods of use. For example, the implantable device can be implanted into the body (e.g., subcutaneously) or the implantable device can be inserted into the body (e.g., intravaginally). The device may be implanted using standard techniques. The delivery route may be intrapulmonary, gastroenteral, subcutaneous, intramuscular, intravaginal, intrauterine, or any other suitable delivery route. The device may be placed in a tissue site of a patient in, on, adjacent to, or near an area of the body where delivery is targeted. The device may also be employed together with current systemic active ingredients. The device can also be employed after a patient has been treated with a therapy to ameliorate post-treatment symptoms or side effects. The implantable device can be in different forms, such as an implant (e.g., subcutaneous implant), an intrauterine system (IUS) (e.g., intrauterine device), a helical coil, a spring, a rod, a cylinder, and/or a vaginal ring. For example, single device including the release structure, the septum and the backing layer can be in the form a ring such that the reservoir is likewise in the form of a ring.

These and other modifications and variations of the present disclosure may be practiced by those of ordinary skill in the art, without departing from the spirit and scope of the present disclosure. In addition, it should be understood that aspects of the various embodiments may be interchanged both in whole or in part. Furthermore, those of ordinary skill in the art will appreciate that the foregoing description is by way of example only, and is not intended to limit the disclosure so further described in such appended claims.

What is claimed is:

1. An implantable and refillable device for delivering a drug compound, the device comprising:
a reservoir within which the drug compound is capable of being retained, wherein the reservoir defines a first surface and a second surface opposing the first surface;
a release structure comprising a hydrophobic polymer and surrounding at least a portion of the reservoir, wherein the release structure is in communication with the reservoir such that the drug compound can pass from the reservoir through the release structure;
a septum positioned adjacent to the first surface of the reservoir; and
a backing layer positioned adjacent to the second surface of the reservoir.

2. The implantable and refillable device of claim 1, wherein the device has a generally circular cross-section.

3. The implantable and refillable device of claim 1, wherein the reservoir has a volume of from about 0.5 to about 5 milliliters.

4. The implantable and refillable device of claim 1, wherein the device is in the form of a disc.

5. The implantable and refillable device of claim 1, wherein the hydrophobic polymer comprises a semi-crystalline olefin copolymer.

6. The implantable and refillable device of claim 5, wherein the semi-crystalline copolymer is derived from at least one olefin monomer and at least one polar monomer.

7. The implantable and refillable device of claim 6, wherein the olefin monomer includes ethylene.

8. The implantable and refillable device of claim 6, wherein the polar monomer includes vinyl acetate, vinyl alcohol, maleic anhydride, maleic acid, acrylic acid, methacrylic acid, acrylate, methacrylate, ethyl acrylate, methyl methacrylate, ethyl methacrylate, or a combination thereof.

9. The implantable and refillable device of claim 6, wherein the polar monomer constitutes from about 10 wt.% to about 45 wt.% of the copolymer.

10. The implantable and refillable device of claim 5, wherein the olefin copolymer includes an ethylene vinyl acetate copolymer.

11. The implantable and refillable device of claim 1, wherein the hydrophobic polymer has a melt flow index of from about 0.2 to about 100 grams per 10 minutes as determined in accordance with ASTM D1238-13 at a temperature of 190° C. and a load of 2.16 kilograms.

12. The implantable and refillable device of claim 1, wherein the release structure further includes a hydrophilic compound.

13. The implantable and refillable device of claim 12, wherein the hydrophilic compound includes a hydrophilic polymer.

14. The implantable and refillable device of claim 12, wherein the hydrophilic compound includes a plurality of water-soluble particles distributed within the release structure.

15. The implantable and refillable device of claim 1, wherein the septum comprises a material that exhibits a Shore A hardness of about 40 or less.

16. The implantable and refillable device of claim 1, wherein the septum comprises a nitrile rubber, styrene block copolymer, polyurethane, silicone elastomer, or a combination thereof.

17. The implantable and refillable device of claim 1, wherein the septum comprises multiple layers.

18. The implantable and refillable device of claim 1, wherein the backing layer includes a puncture-resistant material comprising a polymeric material, metal, ceramic, or a combination thereof.

19. The implantable and refillable device of claim 18, wherein the backing layer comprises biocompatible fibers.

20. The implantable and refillable device of claim 1, wherein the drug compound includes one or more bisphosphonates.

21. The implantable and refillable device of claim 1, wherein the drug compound includes one or more corticosteroids.

22. The implantable and refillable device of claim 1, wherein the drug compound includes one or more selective estrogen receptor modulators.

23. The implantable and refillable device of claim 1, wherein the drug compound includes one or more aromatase inhibitors.

24. The implantable and refillable device of claim 1, wherein the drug compound includes one or more antipsychotics.

25. The implantable and refillable device of claim 1, wherein the drug compound includes one or more nucleic acids.

26. The implantable and refillable device of claim 25, wherein the one or more nucleic acids includes DNA, RNA, TNA, GNA, PNA, or combinations thereof.

27. The implantable and refillable device of claim 26, wherein the one or more nucleic acids includes a mRNA, tRNA, siRNA, snRNA, or combinations thereof.

28. The implantable and refillable device of claim 1, wherein the drug compound comprises one or more chemotherapeutic agents.

29. The implantable and refillable device of claim 1, wherein the drug compound comprises one or more monoclonal antibodies.

30. The implantable and refillable device of claim 29, wherein the one or more monoclonal antibodies includes an anti-PD-1 antibody, anti-PD-L1 antibody, anti-CLTA-4 antibody, anti-HER2 antibody, anti-VEGF antibody, and anti-cKIT antibody, anti-4-1BB antibody, or a combination thereof.

31. The implantable and refillable device of claim 29, wherein the one or more antibodies includes an antibody drug conjugate (ADC).

32. The implantable and refillable device of claim 29, wherein the one or more antibodies includes a multispecific antibody.

33. The implantable and refillable device of claim 29, wherein the one or more antibodies includes an antibody fragment.

34. The implantable and refillable device of claim 29, wherein the one or more antibodies includes an immunocytokine.

35. The implantable and refillable device of claim 29, wherein the one or more antibodies includes an antibody-small interfering RNA (siRNA) conjugate (ARC).

36. A method for prohibiting and/or treating a condition, disease, and/or cosmetic state of a patient, the method comprising implanting the device of independent claim 1 in a patient and filling the reservoir with a drug compound.

37. The method of claim 36, the method of filling the reservoir comprising:
inserting a needle through the septum;
injecting the drug compound into the reservoir through the needle; and
retracting the needle from the septum.

38. The method of claim 36, further comprising, following a period of time during which the drug compound passes across the release structure, refilling the reservoir.

39. The method of claim 36, wherein the device is implanted as an intrauterine implant or a subcutaneous implant.
